SEQUENCE LISTING (20 sequences)

SEQ ID NO: 1 - DNA, 33 bp, synthetic construct
  source 1..33: mol_type = other DNA
cgggcaagtcagagcattagcagctatttaaat

SEQ ID NO: 2 - DNA, 21 bp, synthetic construct
  source 1..21: mol_type = other DNA
gctgcatccagtttgcagtca

SEQ ID NO: 3 - DNA, 27 bp, synthetic construct
  source 1..27: mol_type = other DNA
caacagagtcattcggtgtcattcact

SEQ ID NO: 4 - DNA, 21 bp, synthetic construct
  source 1..21: mol_type = other DNA
ggattcacttttagcaactat

SEQ ID NO: 5 - DNA, 18 bp, synthetic construct
  source 1..18: mol_type = other DNA
agcggaagtggtggtgcc

SEQ ID NO: 6 - DNA, 54 bp, synthetic construct
  source 1..54: mol_type = other DNA
gatcggctcattatggctacggttcggggaccctattactacggtatggacgtc

SEQ ID NO: 7 - protein, 11 aa, synthetic construct
RASQSISSYLN

SEQ ID NO: 8 - protein, 7 aa, synthetic construct
AASSLQS

SEQ ID NO: 9 - protein, 9 aa, synthetic construct
QQSHSVSFT

SEQ ID NO: 10 - protein, 7 aa, synthetic construct
GFTFSNY

SEQ ID NO: 11 - protein, 6 aa, synthetic construct
SGSGGA

SEQ ID NO: 12 - protein, 18 aa, synthetic construct
DRLIMATVRGPYYYGMDV

SEQ ID NO: 13 - DNA, 54 bp, synthetic construct
  source 1..54: mol_type = other DNA
ggcagcaccagcggcagcggcaaaccgggttctggtgaaggctcaacaaagggt

SEQ ID NO: 14 - DNA, 66 bp, synthetic construct
  source 1..66: mol_type = other DNA
atggctctacccgtgacagctttacttttacctctggccttgctgctacatgccgccaggccctct

SEQ ID NO: 15 - DNA, 234 bp, synthetic construct
  source 1..234: mol_type = other DNA
ggtaccgctaaacctaccacaacgccggctcctcgaccaccaacacctgcacctactatcgcttctcaacccctgtccctgagaccagaagcttgccggccagctgctggaggtgcagtgcatacgcggggtctggattttgcctgtgatatatatatctgggccccgttggcagggacttgtggagtccttttattgtcactggtaattacattatattgcaatcatcggaat

SEQ ID NO: 16 - DNA, 459 bp, synthetic construct
  source 1..459: mol_type = other DNA
aggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 17 - DNA, 462 bp, synthetic construct
  source 1..462: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - DNA, 711 bp, synthetic construct
  source 1..711: mol_type = other DNA
atggtcagcaaaggcgaagaagacaacatggcatcactgccagcgacacacgaacttcatattttcggctccatcaatggtgtggacttcgatatggttggacagggtactgggaaccctaacgatggatatgaggagttgaacttgaagagtaccaagggcgatcttcagttttcaccttggattctcgtacctcatataggttatggctttcatcaatatctgccctatcctgacggtatgtcacctttccaagcagctatggttgacgggagcggttatcaggttcaccggacaatgcagtttgaagatggagcctctttgaccgtaaattatcggtatacttatgagggcagccatattaaaggcgaggctcaagttaaggggactgggttccccgcggatggtcccgtaatgacaaacagtctgacggctgcagactggtgccgctctaagaagacatatccaaatgacaagacgattattagcactttcaagtggtcatacactacaggaaatggtaaacgctataggagcactgcccggaccacgtatactttcgcgaagccgatggcggcaaattatttgaaaaaccaaccgatgtatgtgttcagaaagacagaactcaagcattctaaaactgaactgaacttcaaagagtggcagaaagcctttactgatgtaatgggcatggacgaactctataagtaa

SEQ ID NO: 19 - DNA, 63 bp, synthetic construct
  source 1..63: mol_type = other DNA
ggatccggcgagggcaggggaagtcttctaacatgcggggacgtggaggaaaatcccgggccc

SEQ ID NO: 20 - DNA, 2302 bp, synthetic construct
  source 1..2302: mol_type = other DNA
tcgaaagcttggcgcgccatatctaggttagtctttttttctgtggcttctgtctctggttttgtgcttagaaagtcctttcctacttgaaaatgagataaatgttcacctatgttggcttctagtctcttttatggcttcattttttccatttactatagaggttaagagtgtgggtactggagccagactgtctgggacaaacccagcgtcaccccaagccctatgtgtgatttttagccaggcacttaacctctccatccaagaagggccaggtcttcagagctaggggcttgtcatagtggccagatggacatcacctaccacatccaccagcacccatgtcaccccacctgggccaagcctgctgcaggacagggcagccagttctcggaacgaaacctgtggggtggggtatctgccctcttctcttcctccgtggtgtcgatgaagcccggcgcatccggccgccatgacgtcaatggcggaaaaatctgggcaagtcgggggctgtgacaacagggcccagatgcagaccccgatatgaaaacataatctgtgtcccagaaacatcccccattcagcttctgagaaacccagtcagaaagggacgtcccaacagaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatcctcccatccacacatagagcttcagattctctttctttccccagagaccctcaaatatcctctcactcacagaatggtgtctctgcctgcctcgggttggccctgtgatttattttagttcttttcccttgttttttttttttcaaactctatacacttttgttttaaaaactgtggtttctcatgagccctattatctcattgatacctctcacctctgtggtgaggggaagaaatcatattttcagatgactcgtaaagggcaaagaaaaaaacccaaaatttcaaaatttccgtttaagtctcataatcaagaaaaggagaaacacagagagagagaaaaaaaaaactatgagaacccctccccaccccgtgattatcagcgcacacactcatcgaaaaaaatttggattattagaagagagaggtctgcggcttccacaccgtacagcgtggtttttcttctcggtataaaagcaaagttgtttttgatacgtgacagtttcccacaagccaggctgatccttttctgtcagtccacttcaccaagcctgcccttggacaaggacccgtgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccgtcgacgagctcgtatc

(19) United States
(12) Patent Application Publication
(10) Pub. No.: US 2025/0177528 A1
(43) Pub. Date: Jun. 5, 2025

(54) REGULATORY T CELLS WITH CHIMERIC ANTIGEN RECEPTOR TARGETING CO-STIMULATORY MOLECULES TO PREVENT AND/OR TREAT INFLAMMATORY CONDITIONS

(71) Applicant: THE CHILDREN'S MEDICAL CENTER CORPORATION, Boston, MA (US)

(72) Inventors: Leslie S. KEAN, Jamaica Plain, MA (US); Xianliang RUI, Belmont, MA (US); Victor TKACHEV, Natick, MA (US); Ulrike GERDEMANN, Boston, MA (US)

(73) Assignee: THE CHILDREN'S MEDICAL CENTER CORPORATION, Boston, MA (US)

(21) Appl. No.: 18/842,076

(22) PCT Filed: Feb. 28, 2023
(86) PCT No.: PCT/US2023/014032

§ 371 Date: Aug. 28, 2024

Related U.S. Application Data
(60) Provisional application No. 63/314,789, filed on Feb. 28, 2022.

(51) Int. Cl. A61K 40/31; A61K 35/17; A61K 40/11; A61K 40/42; C07K 14/705; C07K 14/725; C07K 16/28; C12N 5/0783

(52) U.S. Cl. CPC A61K 40/31; A61K 35/17; A61K 40/11; A61K 40/4232; C07K 14/7051; C07K 14/70521; C07K 14/70596; C07K 16/2875; C12N 5/0637; A61K 2239/13; A61K 2239/21; A61K 2239/22; C07K 2317/565; C07K 2317/622; C12N 2510/00

(57) ABSTRACT

Described herein are methods and compositions for regulatory T cells (Tregs) that are modified to express a chimeric antigen receptor (CAR) that targets OX40L. Aspects of the invention relate to administering these modified Tregs to a subject having an inflammatory or autoimmune condition.

### CROSS-REFERENCE TO RELATED APPLICATIONS

This application claims benefit under 35 U.S.C. § 119(e) of U.S. Provisional Application No. 63/314,789 filed Feb. 28, 2022, the contents of which are incorporated herein by reference in their entirety.

### GOVERNMENT SUPPORT

This invention was made with government support under Grant Numbers HL095791 and AI051731 awarded by the National Institutes of Health. The government has certain rights in the invention.

### TECHNICAL FIELD

The technology described herein relates to chimeric antigen receptor (CAR) constructs, e.g., in regulatory T cells (Tregs) targeted to co-stimulatory molecules.

### BACKGROUND

The major cause of inflammatory conditions or autoimmunity as well as transplant rejection following an organ transplant or graft-versus-host disease (GVHD) following a hematopoietic stem cell transplant (HCT), is autoreactive and alloreactive T cells attacking the patient's or donor's organ(s) or tissue. Current treatments for preventing autoimmunity, transplant rejection, and GVHD involve stringent immunosuppressant therapy, which in itself can lead to severe, unwanted side effects. In order to prevent autoimmunity, transplant rejection, and GVHD without inducing unwanted side effects, it would be advantageous to be able to utilize engineered T cells, in particular engineered regulatory T cells (Tregs), to treat a patient receiving an organ transplant.

Tregs suppress immune responses of other cells. These cells inhibit T cell proliferation and cytokine production and can prevent autoimmunity or shut down immune responses after successfully eliminating invading organisms. They come in several forms with the most well-understood being those that express CD4, CD25, Foxp3, and Helios (CD4⁺CD25⁺ Tregs). Engineering Tregs and directing Tregs toward appropriate targets can enhance suppression of aberrant immune responses.

### SUMMARY

As described herein, chimeric antigen receptors (CARs) offer a promising method of targeting APCs and the OX40L co-stimulatory molecules. CARs are engineered receptor proteins, in which the natural recognition portion is removed and replaced with a specific recognition portion that redirects them to target and bind a specific protein. A T cell expressing a CAR is referred to as a CAR-T. CARs and CAR-Ts are most often used to treat tumors and cancers, and to a lesser extent to treat inflammatory conditions, but there remains a need for more effective CARs to minimize or eliminate the undesirable immune response to the underlying causes of inflammatory conditions and autoimmune disorders. Generation of OX40L CAR-Treg aims to provide more constant blockade of cytokine production during inflammatory or autoimmune triggers. Compared to a few of the newly developed CAR-Tregs targeting specific antigens, such as HLA-A2, CEA, and CD83, CAR-Tregs targeting OX40L cover a larger spectrum of inflammatory conditions, providing broader applications. Moreover, activated Tregs help restore immune tolerance and maintain conventional T cell (Tcon) and Treg functional balance, rendering OX40L CAR Treg cells as an alternative or superior agent than antibodies for treatment of inflammatory conditions or autoimmune diseases.

As described herein, the inventors have produced human regulatory T cells (Treg) that are engineered to express a chimeric antigen receptor (CAR) that specifically targets OX40L. In some embodiments, described herein is a CAR comprising an extracellular recognition portion comprising an anti-OX40L antibody reagent, e,g., an scFv, and an intracellular signaling region(s) to permit or induce CAR-Treg cell activation upon binding to OX40 ligand on APCs. Furthermore, in order to restrict the harmful effects from Tcon activation contamination during purification and expansion of Treg cells, a synthetic FoxP3 promoter drives the CAR expression. This promoter limits expression of the CAR to Treg cells. Accordingly, these CAR-Tregs and related compositions can be used in therapeutic methods to treat diseases described herein.

In one aspect of any of the embodiments, described herein is a chimeric antigen receptor (CAR) comprising, from N-terminus to C-terminus: (a) an extracellular recognition portion that specifically binds to OX40L; (b) a transmembrane portion; and (c) an intracellular signaling portion.

In some embodiments of any of these aspects, the C-terminal of the intracellular signaling portion of the CAR further comprises a detectable polypeptide.

In some embodiments of any of these aspects, the detectable polypeptide of the CAR is a fluorescent polypeptide.

In some embodiments of any of these aspects, the fluorescent polypeptide of the CAR is NeonGreen.

In some embodiments of any of these aspects, the CAR further comprises a cleavage site between the intracellular signaling portion and the detectable polypeptide.

In some embodiments of any of these aspects, the cleavage site of the CAR is a cleavable T2A site or a tandem P2A-T2A site.

In some embodiments of any of these aspects, the recognition portion of the CAR is an antibody reagent or ligand functional domain.

In some embodiments of any of these aspects, the antibody reagent of the CAR is a scFV.

In some embodiments of any of these aspects, the antibody reagent of the CAR is an anti-OX40L antibody reagent.

In some embodiments of any of these aspects, the antibody reagent of the CAR comprises CDR sequences at least 80% identical, at least 85% identical, at least 90% identical, at least 95% identical, or 100% identical to the six CDRs of SEQ ID NOs: 1-6.

In some embodiments of any of these aspects, the antibody reagent of the CAR comprises a sequence at least 80% identical, at least 85% identical, at least 90% identical, at least 95% identical, or 100% identical to the amino acid sequences of SEQ ID NO 7-12.

In some embodiments of any of these aspects, the intracellular signaling portion of the CAR comprises one or more of: a CD28 co-signaling domain, a 41BB co-signaling domain, a IL2Rα JAK3 and IL2Rβ STAT5 composite docking site, a TGFβ-R SMAD2/3 docking site, and a CD3zeta signaling domain.

In some embodiments of any of these aspects, the intracellular signaling portion of the CAR comprises a CD28 co-signaling domain and a CD3zeta domain.

In some embodiments of any of these aspects, the intracellular signaling portion of the CAR comprises a 41BB co-signaling domain and a CD3zeta domain.

In some embodiments of any of these aspects, is a nucleic acid molecule encoding a CAR.

In some embodiments of any of these aspects, the expression of a nucleic acid molecule is controlled by a Treg-specific promoter or a MND promoter.

In some embodiments of any of these aspects, the Treg-specific promoter of the nucleic acid molecule encoding a CAR comprises a FoxP3 promoter or a FoxP3 and IKZF2/Helios hybrid promoter.

In some embodiments of any of these aspects, is a vector comprising a nucleic acid molecule encoding the CAR.

In some embodiments of any of these aspects, is a cell comprising a CAR, or the nucleic acid molecule encoding the CAR, or a vector comprising the nucleic acid molecule encoding the CAR.

In some embodiments of any of these aspects, the cell the cell is a Treg.

In some embodiments of any of these aspects, the Treg expresses Foxp3.

In some embodiments of any of these aspects, the Treg expresses CD4 and CD25.

In some embodiments of any of these aspects, is a population of cells, at least 80% of which are cells of any of these aspects.

In some embodiments of any of these aspects, is a method of treating an autoimmune or inflammatory condition in a subject in need thereof, the method comprising administering to the subject a cell or population of cells of any of these aspects.

In some embodiments of any of these aspects, the autoimmune or inflammatory condition comprises an allograft or xenograft rejection, or graft-vs. host disease (GVHD).

In some embodiments of any of these aspects, the autoimmune or inflammatory condition is selected from the group consisting of: an inflammatory bowel disease; rheumatoid arthritis; type I diabetes mellitus or autoimmune insulitis; multiple sclerosis; autoimmune thyroiditis; autoimmune gastritis; autoimmune uveitis or uveoretinitis; autoimmune orchitis; autoimmune oophoritis; psoriasis; vitiligo; autoimmune prostatitis; any undesired immune response; tissue rejection; and an inflammatory condition.

In some embodiments of any of these aspects, the population of Tregs are autologous to the subject.

In some embodiments of any of these aspects, the population of Tregs are allogenic to the subject.

In some embodiments of any of these aspects, a cell or population of cells for use in a method of treating an autoimmune or inflammatory condition in a subject in need thereof, comprises administering the cell or population of cells to the subject.

In some embodiments of any of these aspects, autoimmune or inflammatory condition comprises an allograft or xenograft rejection, or graft-vs. host disease (GVHD).

In some embodiments of any of these aspects, the autoimmune or inflammatory condition is selected from the group consisting of: an inflammatory bowel disease; rheumatoid arthritis; type I diabetes mellitus or autoimmune insulitis; multiple sclerosis; autoimmune thyroiditis; autoimmune gastritis; autoimmune uveitis or uveoretinitis; autoimmune orchitis; autoimmune oophoritis; psoriasis; vitiligo; autoimmune prostatitis; any undesired immune response; tissue rejection; and an inflammatory condition.

In some embodiments of any of these aspects, the population of Tregs are autologous to the subject.

In some embodiments of any of these aspects the population of Tregs are allogenic to the subject.

### BRIEF DESCRIPTION OF THE DRAWINGS

FIGS. 1A-1D show construction and expansion of OX40L CAR-Treg cells (FIG. 1A) Schema of the Treg-specific Chimeric Antigen Receptor (CAR)-encoding construct: a single-chain Fragment variant of derived from antigen-binding fragment of anti-OX40L-antibody was linked to intracellular CD3zeta and 4-1BB or CD28 signaling domains, and, via cleavable T2A site to fluorescent protein reporter NeonGreen. Expression of the CAR and fluorescent reporter protein was controlled by either Treg-specific promoter or synthetic non-specific MND promoter. Constructs encoding NeonGreen only under Treg-specific or MND promoter were used as controls. (FIG. 1B) Demonstration of cell-surface expression of OX40L-specific CAR construct: Binding of OX40L soluble protein to cell surface of Jurkat lymphoid cells in vitro was observed in >85% of NeonGreen-positive cells (that successfully transduced with CAR/reporter-encoding construct), whereas cells that were transduced with NeonGreen-encoding construct demonstrated only negligible binding of OX40L protein. (FIG. 1C) Human FACS-purified CD25+CD127− Treg cells were transduced with OX40L-specific CAR constructs with signaling CD3zeta/4-1BB (BBz) or CD3zeta/CD28 (28z) domains under control of FoxP3-specific promoter and then were expanded in vitro using CD3/CD28 microbeads and IL-2. The bar graph depicts the normal expansion of CAR-transduced Treg cells. (FIG. 1D) Expanded human Treg cells stably express FoxP3-promoter-controlled CAR construct. Human FACS-purified CD25+CD127− Treg and CD25−CD127+ conventional CD4 (Tconv) cells were transduced with OX40L-specific CAR constructs with signaling CD3zeta/4-1BB (BBz) or CD3zeta/CD28 (28z) domains under control of FoxP3-specific promoter and then were expanded in vitro using CD3/CD28 microbeads and IL-2. The percentage of NeonGreen+ cells in Treg and Tconv cell cultures was assessed by flow cytometry at different time points. The graph demonstrates the stable expression of FoxP3-promoter-controlled CAR construct in Treg cells whereas in resting Tconv cells expression of CAR-construct was decreased upon CD3/CD28 beads removal (D18).

FIGS. 2A-2D show large-scale expansion of OX40L CAR-Tregs ex vivo. (FIG. 2A) Experimental schema of the large-scale CAR-Treg cells expansion experiment. (FIG. 2B) The efficacy of the lentivirus transduction procedure: human FACS-purified CD25+CD127− Treg cells and CD25−CD127+ Tconv cells were transduced with lentivirus containing genetic constructs encoding NeonGreen reporter fluorescent protein or CAR fused with NeonGreen reporter (see FIG. 1A). The percentage of NeonGreen+ cells was measured by flow cytometry on day 13 in Treg and Tconv cultures. (FIG. 2C) Large-scale expansion of CAR-Treg cells ex vivo: Untransduced human Treg cell, or Treg cell transduced with NeonGreen reporter or CAR construct were expanded ex vivo for 34 days. The plot demonstrates the similar expansion capacity of CAR-expressing Treg cells. (FIG. 2D) Expanded ex vivo human CAR-Treg cells demonstrate stable FoxP3 expression. Human FACS-purified CD25+CD127− Treg cells were transduced with BBz or 28z CAR constructs or with NeonGreen reporter and were expanded ex vivo using CD3/CD28+IL-2. The expression of FoxP3 protein was measured by flow cytometry and confirms FoxP3 expression in CAR-Treg cell at the same level as in endogenous Treg control cells.

FIGS. 3A-3C show functional characteristics of CAR-Treg cells. (FIG. 3A) CAR stimulation induces expression of Treg activation/suppression markers: Treg cells transduced with BBz or 28z CAR construct were stimulated in vitro via CAR by co-culture with K562 cells expressing OX40L (K562-L) or via endogenous TCR by co-culture with CD3/CD28 activating beads. Anti-OX40 antibodies were added to the Treg/K562-L cultures (K562-L+Ab) to block potential signaling via endogenous OX40L/OX40 pathway and, thus, to confirm that CAR-Treg cells receives stimulation through CAR. The plot demonstrates CAR-mediated induction of Treg activation/suppression markers CTLA4, LAP, CD71, GARP, and LAG-3 at the levels comparable to TCR stimulation. (FIG. 3B) CAR stimulation induces production of Treg-supportive cytokine IL-2 but not proinflammatory cytokines: BBz and 28z CAR-expressing Treg cells were stimulated in vitro via CAR (K562-L) or via endogenous TCR (CD3/CD28 beads). Anti-OX40 antibodies were added to the Treg/K562-L cultures to block potential signaling via endogenous OX40L/OX40 pathway. The plot demonstrates CAR-mediated induction of IL-2 production, whereas the production of pro-inflammatory cytokines IFNg-gamma, TNF-alpha and IL-17A remains unaffected. (FIG. 3C) CAR stimulation improves suppressive function of Treg cells in vitro: Control Treg cells and Treg cells expressing BBz or 28z CAR were co-cultured with CD3/CD28-bead-activated conventional CD4 T cells (CD4 Tconv) in the presence or absence of OX40L-expressing K562 cells (K562-L and K562, respectively). Suppressive capacity of Treg cells was assessed using flow cytometry by relative inhibition of CD4 Tconv proliferation. This plot depicts the increased suppressive capacity of CAR-Treg cells co-cultured with OX40L-expressing K562 cells.

FIG. 4 shows proposed suppressive mechanisms for OX40L CAR-Tregs

FIGS. 5A-5C show the effects of blocking OX40L in mouse models of transplantation. (FIG. 5A) Schematic representation of experimental design for evaluating the effect of anti-human OX40L (hOX40L) treatment on CD8⁺ T cell functions. (FIG. 5B) Human CD8⁺ T cells were purified from mouse spleens. % Specific lysis was assessed by measuring fluorescence intensity after 4 h incubation of CD8⁺ T cells and U937 cells labeled with fluorochrome. Error bars represent mean±SD (n=5 mice per group) and data are representative of three independent experiments. Statistical significance was determined with Sidak's two-way ANOVA test. ns, not significant. (FIG. 5C) The clinical score and survival of mice recipients of hPBMCs following treatment with anti-hOX40L.

FIG. 6 shows Anti-OX40L/Sirolimus prophylaxis results in long term GVHD-free survival following allogeneic HCT.

FIG. 7 shows schema of the Treg-specific CAR-encoding construct or NeonGreen encoding control construct.

FIGS. 8A-8B show expression of OX40L-CAR constructs. (FIG. 8A) Schematic for generation of Ox40L CAR-Tregs. (FIG. 8B) Surface expression of FOXP3-driven OX40L-CAR and FOXP3-driven NeonGreen control constructs.

FIGS. 9A-9B show stable and selective expression of OX40L-CAR Tregs. (FIG. 9A) Measurement of FOXP3 positive signal in CD+ cells show similar percentages in Treg, Neon-Treg, and CAR-Tregs demonstrating CAR-Treg stability after ex vivo expansion for 23 days. (FIG. 9B) Measurement of NeonGreen positive signal in CAR-Treg compared to CAR-Tcon demonstrates stable and selective FOXP3 promoter activity in Tregs.

FIGS. 10A-10B show OX40L CAR-Treg effect on Treg suppressive proteins and pro-inflammatory cytokines. (FIG. 10A) Schematic of OX40L specific CAR-Treg activation. (FIG. 10B) After viral transduction, OX40L CAR-Tregs and control Neon-Tregs were co-cultured with irradiated WT K562 cells or OX40L-expressing K562 cells. After 48-72 hrs., expression of the indicated cytokines or suppressive proteins was detected by flow cytometry.

FIGS. 11A-11B show CAR-Tregs suppress T cell expansion in vitro. (FIG. 11A) Schematic of Treg suppression assay setup. (FIG. 11B) Measuring % of CD4 suppression in response to varying Treg:Tresponder ratios shows that activated CAR-Tregs via OX40L engagement more potently suppress T cell expansion in vitro.

FIGS. 12A-12B show CAR-Tregs suppress T cell expansion in MLR. (FIG. 12A) Schematic of MLR suppression assay. (FIG. 12B) Measurement of % of CD4 suppression in response to varying Treg:Tresponder ratios show CAR-Treg suppress MLR compared to Neon-Tregs.

FIGS. 13A-13B show OX40L CAR-Treg effect on mouse model of GVHD. (FIG. 13A) Xenogenetic GVHD experimental setup. (FIG. 13B) Human PBMCs isolated from healthy donors were retro-orbitally injected into sublethally irradiated NSG mice alone or in conjunction with control Neon-Tregs or OX40L CAR-Tregs. Mice were monitored twice weekly for weight loss, GvHD score and survival endpoints. Both Neon-Tregs and OX40L CAR-Tregs delayed the onset of GvHD, but OX40L CAR-Treg demonstrated significantly lower GvHD clinical scores and improved overall survival. ***p<0.001.

### DETAILED DESCRIPTION

Described herein are chimeric antigen receptors (CARs) that are engineered in regulatory T cells (Tregs) and that specifically target a costimulatory molecule, e.g., OX40L. The technology described herein can cause a blockade of OX40-OX40L interaction and be efficacious in many inflammatory conditions, autoimmune diseases, and graft-vs-host disease (GVHD). OX40L CAR-Tregs restore immune tolerance and maintain the functional balance of convention T cells (Tcon) and Tregs, thereby providing an additional, alternative, or superior therapeutic agent for the above-mentioned diseases. Also described herein are polynucleotides and vectors encoding said CAR polypeptides, cells expressing said CAR polypeptides, pharmaceutical compositions comprising said CAR polypeptides, and methods of using said CAR polypeptides.

Accordingly, in one aspect of any of the embodiments, described herein is a chimeric antigen receptor (CAR) polypeptide comprising, from N-terminus to C-terminus:
a. an extracellular recognition portion that binds specifically to one or more co-stimulatory molecules, e.g., OX40L;
b. a transmembrane portion; and
c. an intracellular signaling portion.

CARs are receptor proteins that have been engineered to comprise an exogenous recognition domain that specifically binds a desired target, e.g., OX40L. A cell expressing or comprising such a CAR is thereby provided with the ability to target a desired target. The receptors are chimeric because they combine both antigen-binding and immune cell activating functions into a single receptor in a combination which does not occur in nature, and/or because they combine antigen-binding sequences and immune cell activating sequences from different genetic origins. In some embodiments of any of the aspects, the extracellular recognition portion of a CAR described herein does not naturally occur in a protein that comprises the intracellular signaling portion described herein.

In some embodiments of any of the aspects, a CAR as described herein comprises an extracellular recognition portion. As used herein, “extracellular recognition portion” refers to a polypeptide sequence that binds to a target and will be displayed on the extracellular side of cell membrane when the CAR is expressed in a cell. In some embodiments of any of the aspects, the extracellular recognition portion comprises an antibody, antibody reagent, an antigen-binding fragment thereof, a F(ab) fragment, a F(ab′) fragment, a single chain variable fragment (scFv), or a single-domain antibody (sdAb). In some embodiments of any of the aspects, the extracellular recognition portion is monovalent, bivalent, or multivalent. In some embodiments of any of the aspects, the extracellular recognition portion comprises a human, humanized, or chimeric antibody or antibody reagent.

Antibody reagents are specific for the targets and/or markers described herein, e.g., T-cell co-stimulatory molecules. Such reagents are readily commercially available. In some embodiments of any of the aspects, the extracellular recognition portion can be an antibody reagent comprising one or more (e.g., one, two, three, four, five, or six) CDRs of any one of the antibodies described herein or known in the art. In some embodiments of any of the aspects, an antibody reagent specific for a target and/or marker described herein (e.g., that binds specifically to a T-cell co-stimulatory molecule as described herein) can be an antibody reagent comprising the six CDRs of any one of the antibodies described herein or known in the art. In some embodiments of any of the aspects, an antibody reagent specific for a target and/or marker described herein (e.g., that binds specifically to a T-cell co-stimulatory molecule as described herein) can be an antibody reagent comprising the three heavy chain CDRs of any one of the antibodies described herein or known in the art. In some embodiments of any of the aspects, an antibody reagent specific for a target and/or marker described herein (e.g., that binds specifically to a T-cell co-stimulatory molecule as described herein) can be an antibody reagent comprising the three light chain CDRs of any one of the antibodies described herein or known in the art. In some embodiments of any of the aspects, an antibody reagent specific for a target and/or marker described herein (e.g., that binds specifically to a T-cell co-stimulatory molecule as described herein) can be an antibody reagent comprising the VH and/or VL domains of any one of the antibodies described herein or known in the art. In some embodiments of any of the aspects, an antibody reagent specific for a target and/or marker described herein (e.g., that binds specifically to a T-cell co-stimulatory molecule as described herein) can be an antibody reagent comprising the VH and VL domains of any one of the antibodies described herein or known in the art. Such antibody reagents are specifically contemplated for use in the methods and/or compositions described herein.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). VH refers to the variable domain of the heavy chain. VL refers to the variable domain of the light chain. For the methods and compositions described herein, the amino acid positions assigned to CDRs and FRs may be defined according to Rabat (Sequences of Proteins of Immunological Interest (National Institutes of Health, Bethesda, Md., 1987 and 1991)). Amino acid numbering of antibodies or antigen binding fragments is also according to that of Rabat.

The terms “antigen-binding fragment” or “antigen-binding domain”, which are used interchangeably herein are used to refer to one or more fragments of a full-length antibody that retain the ability to specifically bind to a target of interest (e.g., that binds specifically to a T-cell co-stimulatory molecule as described herein). Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fv fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. The light chain and heavy chain-derived sequences can be provided in N to C terminal order respectively, or in the opposite order.

Co-stimulatory molecules are a group of cell surface molecules that amplify or counteract the initial activating signals provided to T cells from the T cell receptor (TCR) following its interaction with an antigen/major histocompatibility complex (MHC). OX40L is an important costimulatory molecule and is inducibly expressed on professional antigen presenting cells (APCs) such as B-cells, dendritic cells (DCs) and macrophages under inflammatory conditions. OX40L and its binding partner OX40 regulate cytokine production from T cells, antigen-presenting cells, NK cells, and NKT cells. Non-limiting examples of other T-cell co-stimulatory molecules that can be targeted include CD28, CD80, CTLA-4, PDL-1, CD86, ICOS, ICOS-L, PDL-2, CD27, CD70, CD30, CD30L, CD40L, CD40, GITR, GITRL, TIM-1, TIM-2, TIM-3, and TIM-4.

In some embodiments of any of the aspects, the extracellular recognition portion comprises, consists of, or consists essentially of an anti-OX40L antibody, antibody reagent, or antigen-binding portion thereof. OX40L, also known as tumor necrosis factor ligand superfamily member 4 is a gene that encodes a cytokine of the tumor necrosis factor ligand family that mediates adhesion of activated T cells to endothelial cells. The sequence of OX40L is known in the art for a number of species, including human. For example, see the NCBI Database entry for Gene ID No: 7292.

Antibodies and antibody reagents that specifically bind OX40L are known in the art and are commercially available. For example, the following anti-OX40L antibodies are commercially available:
Ab263910 [EPR23155-317] from Abcam;
Ab76130 [EP1168Y] from Abcam;
LS-B10561 [362CT19.3.3] from LSBio;
AB_2207272/Cat. No. 326307 [11C3.1] from BioLegend;
MAD10541 [159403] from RnD Systems;
MA5-37543 [362CT19.3.3] from Invitrogen;
M02554 [OTI5D8] from BoosterBio;
sc-71768 [2Q1716] from Santa Cruz Biotechnology; and
AM01048PU-S [7D6] from Origene.

In some embodiments, described herein is a CAR with an extracellular recognition portion that comprises, consists of, or consists essentially of an OX40L antibody reagent comprising light chain CDRs encoded by the nucleic acid sequences of SEQ ID Nos 1-3 or a conservative substitution variant of such nucleic acid sequence. In some embodiments, described herein is a CAR with an extracellular recognition portion that comprises, consists of, or consists essentially of an OX40L antibody reagent comprising heavy chain CDRs encoded by the nucleic acid sequences of SEQ ID Nos 4-6 or a conservative substitution variant of such nucleic acid sequence.

In some embodiments of any of the aspects, the extracellular recognition domain of a CAR as described herein comprises light chain CDRs encoded by the nucleic acid sequences presented in SEQ ID NOs: 1-3 and heavy chain CDRs encoded by the nucleic acid sequences of SEQ IDs 4-6 or a nucleic acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequences of SEQ ID NOs: 1-3 and SEQ ID NOs: 4-6 that maintains the same functions. In some embodiments of any of the aspects, the CAR comprises at least one heavy or light chain complementary determining region (CDR) encoded by a nucleic acid selected from the group consisting of:
(a) a light chain CDR1 having the nucleic acid sequence of SEQ ID NO: 1;
(b) a light chain CDR2 having the nucleic acid sequence of SEQ ID NO: 2;
(c) a light chain CDR3 having the nucleic acid sequence of SEQ ID NO: 3;
(d) a heavy chain CDR1 having the nucleic acid sequence of SEQ ID NO: 4;
(e) a heavy chain CDR2 having the nucleic acid sequence of SEQ ID NO: 5; and
(f) a heavy chain CDR3 having the nucleic acid sequence of SEQ ID NO: 6.

SEQ ID NO 1: is a nucleic acid sequence that encodes light chain CDR1:

| | |
|---|---|
| cgggcaagtc agagcattag cagctattta aat | 33 |

SEQ ID NO 2: is a nucleic acid sequence that encodes light chain CDR2:

| | |
|---|---|
| gctgcatcca gtttgcagtc a | 21 |

SEQ ID NO 3: is a nucleic acid sequence that encodes light chain CDR3:

| | |
|---|---|
| caacagagtc attcggtgtc attcact | 27 |

SEQ ID NO 4: is a nucleic acid sequence that encodes heavy chain CDR1:

| | |
|---|---|
| ggattcactt ttagcaacta t | 21 |

SEQ ID NO 5: is a nucleic acid sequence that encodes heavy chain CDR2:

| | |
|---|---|
| agcggaagtg gtggtgcc | 18 |

SEQ ID NO 6: is a nucleic acid sequence that encodes heavy chain CDR3:

| | |
|---|---|
| gatcggctca ttatggctac ggttcgggga ccctattact acggtatgga | 50 |
| cgtc | 54 |

In some embodiments, described herein is a CAR with an extracellular recognition portion that comprises, consists of, or consists essentially of an anti-OX40L antibody reagent comprising the light chain CDRs of SEQ ID Nos 7-9 or a conservative substitution variant of such a sequence. In some embodiments, described herein is a CAR with an extracellular recognition portion that comprises, consists of, or consists essentially of an anti-OX40L antibody reagent comprising the heavy chain CDRs of SEQ ID Nos 10-12 or a conservative substitution variant of such nucleic acid sequence.

In some embodiments of any of the aspects, the extracellular recognition portion of a CAR as described herein comprises light chain CDRs having the amino acid sequences presented in SEQ ID NOs: 7-9 and heavy chain CDRs having the amino acid sequences presented in SEQ IDs 10-12 or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NOs: 7-9 and SEQ ID NOs: 10-12 that maintains the same functions. In some embodiments of any of the aspects, the CAR comprises at least one heavy or light chain complementary determining region (CDR) selected from the group consisting of:
(g) a light chain CDR1 having the amino acid sequence of SEQ ID NO: 7;
(h) a light chain CDR2 having the amino acid sequence of SEQ ID NO: 8;
(i) a light chain CDR3 having the amino acid sequence of SEQ ID NO: 9;
(j) a heavy chain CDR1 having the amino acid sequence of SEQ ID NO: 10;
(k) a heavy chain CDR2 having the amino acid sequence of SEQ ID NO: 11; and
(l) a heavy chain CDR3 having the amino acid sequence of SEQ ID NO: 12.

SEQ ID NO 7: is an amino acid sequence of light chain CDR1:

| | |
|---|---|
| RASQSISSYL N | 11 |

SEQ ID NO 8: is an amino acid sequence of light chain CDR2:

| | |
|---|---|
| AASSLQS | 7 |

SEQ ID NO 9: is an amino acid sequence of light chain CDR3:

| | |
|---|---|
| QQSHSVSFT | 9 |

SEQ ID NO 10: is an amino acid sequence of heavy chain CDR1:

| | |
|---|---|
| GFTFSNY | 7 |

SEQ ID NO 11: is an amino acid sequence of heavy chain CDR2:

| | |
|---|---|
| SGSGGA | 6 |

SEQ ID NO 12: is an amino acid sequence of heavy chain CDR3:

| | |
|---|---|
| DRLIMATVRG PYYYGMDV | 18 |

A VH domain and a VL domain of an antibody can be joined by a peptide linker to form a VH/VL single chain antigen binding domain (e.g., as an scFv). As used herein, the term “linker” refers to a chemical or peptide structure that covalently joins two polypeptide moieties. Lengths of linkers can be varied to modify the ability of linked domains to form, e.g., intramolecular or intermolecular dimers. For example, a diabody includes a short linker peptide between VH and VL domains, usually 5 amino acids, that will not permit the VH and VL domains to pair to form an antigen-binding domain; expression of two different VH-VL constructs with this short linker arrangement in a cell permits the VH domain of a first VH-VL polypeptide chain to dimerize with the VL domain of the second VH-VL polypeptide chain, and the corresponding VL domain of the first VH-VL polypeptide chain to dimerize with the VH domain of the second VH-VL polypeptide chain, thereby generating a bispecific construct. In contrast, when the VH and VL domains are separated by a longer peptide linker, most often 15-20 amino acids, the VH domain and the VL domain on the same polypeptide chain can dimerize to form an scFv.

In some embodiments of any of the aspects, a CAR as described herein comprises a linker domain between the VL and VH domains of the extracellular recognition portion. In some embodiments of any of the aspects, the linker domain comprises, consists of, or consists essentially of the polypeptide encoded by SEQ ID NO: 13 or a polypeptide that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the polypeptide encoded by SEQ ID NO: 13.

In some embodiments of any of the aspects, the linker domain is encoded by a nucleic acid sequence presented in SEQ ID NO: 13 or a nucleic acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of SEQ ID NO: 13.

SEQ ID NO: 13 is a nucleic acid sequence that encodes a CAR linker domain.

| | |
|---|---|
| ggcagcacca gcggcagcgg caaaccgggt tctggtgaag gctcaacaaa | 50 |
| gggt | 54 |

In addition to scFvs, non-antibody-based approaches have also been used to direct CAR specificity, usually taking advantage of the ligand functional domain. As used herein, “ligand functional domain” is the portion of a molecule that binds to another molecule. In some embodiments of any of the aspects, the polypeptide described herein can comprise cytokines, innate immune receptors, TNF receptors, growth factors, and structural proteins, which have all been successfully used as CAR extracellular recognition portion. In some embodiments of any of the aspects, natural ligand functional domains that can direct CAR specificity in the polypeptide described herein, include NKp30, NKG2D, DNAM-1, CD27, CD16, GM-CSF, Adnectin, IL-13, IL-11, FSH, and TIE.

In some embodiments of any of the aspects, a CAR as described herein further comprises a leader sequence on the N-terminus of the extracellular recognition portion. As used herein, the term “leader sequence” refers to an amino-terminal sequence comprising or consisting of a signal peptide. The signal peptide typically consists of 13 to 36 rather hydrophobic amino acids. Signal peptides have a common structure: a short, positively charged amino-terminal region (n-region); a central hydrophobic region (h-region); and a more polar carboxy-terminal region (c-region) containing the site that is cleaved by the signal peptidase. On the ER luminal side, the signal peptide is cleaved off by a signal peptidase. After successful folding of the nascent polypeptide by ER resident chaperones and foldases, the protein is further directed to exit the ER. This process may be supported by the presence of an N-terminal pro-sequence.

In some embodiments of any of the aspects, the leader sequence encodes a CD8a signal peptide. In some embodiments, the leader sequence of a CAR as described herein is encoded by a nucleic acid sequence presented in SEQ ID NO: 14 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 14 that maintains the same function or a codon-optimized version of SEQ ID NO: 14. In some embodiments of any of the aspects, the leader sequence comprises, consists of, or consists essentially of the polypeptide encoded by SEQ ID NO: 14 or a polypeptide that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the polypeptide encoded by SEQ ID NO: 14.

SEQ ID NO: 14 is a nucleic acid sequence that encodes a CAR leader sequence

| | |
|---|---|
| atggctctac ccgtgacagc tttactttta cctctggcct tgctgctaca | 50 |
| tgccgccagg ccctct | 66 |

The CAR polypeptides described herein comprise a transmembrane portion. The transmembrane portion is a structural component, typically comprising a hydrophobic alpha helix that spans the cell membrane. It anchors the CAR to the plasma membrane, bridging the extracellular hinge and/or recognition portion with the intracellular signaling portion. This domain promotes stability of the receptor as a whole. Transmembrane structures and sequences are well known in the art, e.g., for any transmembrane protein with annotation in the PDB or NCBI databases. With respect to the transmembrane portion, the CAR can be designed to comprise a transmembrane portion that is fused to the extracellular recognition of the CAR. In some embodiments of any of the aspects, the transmembrane portion that naturally is associated with one of the other portions in the CAR is used. In some instances, the transmembrane portion can be selected or modified by amino acid substitution to avoid binding of such portions to the transmembrane portions of the same or different surface membrane proteins to minimize interactions with other members of the receptor complex.

In some embodiments of any of the aspects, the transmembrane portion can be derived either from a natural or from a synthetic source. Where the source is natural, the transmembrane portion can be the transmembrane portion of any membrane-bound or transmembrane protein or derived therefrom. Transmembrane portions of particular use for the CAR polypeptide described herein (i.e. comprise at least the transmembrane region(s) of) the alpha, beta or zeta chain of the T-cell receptor, CD28, CD23zeta, CD28, 4-1BB, CD3 epsilon, CD45, CD4, CD5, CD8, CD9, CD16, CD22, CD33, CD37, CD64, CD80, CD86, CD134, CD137, CD154. Alternatively, the transmembrane portion can be synthetic, in which case it will comprise predominantly hydrophobic residues such as leucine and valine. Preferably a triplet of phenylalanine, tryptophan and valine will be found at each end of a synthetic transmembrane portion. Optionally, a short oligo- or polypeptide linker, preferably between 2 and 10 amino acids in length may form the linkage between the transmembrane portion and the intracellular signaling portion of the CAR. A glycine-serine doublet provides a particularly suitable linker. In some embodiments of any of the aspects, the transmembrane portion comprises the transmembrane portion of CD28. In some embodiments of any of the aspects, the transmembrane domain comprises the transmembrane portion of CD8.

The CAR can further comprise a hinge region between said extracellular recognition portion and said transmembrane portion. The term “hinge region” used herein generally means any oligo- or polypeptide that functions to link the transmembrane portion to the extracellular recognition portion. In particular, hinge regions are used to provide more flexibility and accessibility for the extracellular recognition portion. A hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region can be a synthetic sequence that corresponds to a naturally occurring hinge sequence, or may be an entirely synthetic hinge sequence.

In some embodiments of any of the aspects, the hinge domain and transmembrane portion of a CAR as described herein comprise, consist of, or consist essentially of a polypeptide sequence encoded by the nucleic acid sequence presented in SEQ ID NO: 15 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 15 that maintains the same function or a codon-optimized version of SEQ ID NO: 15.

SEQ ID NO: 15 is a nucleic acid sequence that encodes a CAR hinge and transmembrane portion:

| | |
|---|---|
| ggtaccgcta aacctaccac aacgccggct cctcgaccac caacacctgc | 50 |
| acctactatc gcttctcaac ccctgtccct gagaccagaa gcttgccggc | 100 |
| cagctgctgg aggtgcagtg catacgcggg gtctggattt tgcctgtgat | 150 |
| atatatatct gggccccgtt ggcagggact tgtggagtcc ttttattgtc | 200 |
| actggtaatt acattatatt gcaatcatc ggaat | 235 |

In some embodiments of any of the aspects, a CAR as described herein comprises an intracellular signaling portion. As used herein, “intracellular signaling portion” refers to a polypeptide sequence that comprises one or more TCR signaling domains and will be displayed on the intracellular space of a cell membrane when the CAR is expressed in a cell. After an antigen is bound to the extracellular recognition portion, CAR receptors cluster together and transmit an activation signal and the intracellular signaling portion of the receptor perpetuates signaling inside the T cell. Normal T cell activation via TCR signaling relies on the phosphorylation of immunoreceptor tyrosine-based activation motifs (ITAMs) present in the intracellular domain of CD3-zeta. To mimic this process, CD3-zeta's intracellular domain is commonly used as part of the intracellular signaling portion of the CARs described herein. In some embodiments of any of the aspects, any ITAM-containing domains can be used in the intracellular signaling portion of the CAR polypeptides as described herein. Examples of ITAM containing primary cytoplasmic signaling sequences that are of particular use in the CAR polypeptides described herein include those derived from TCR zeta (also referred to as the zeta-chain, CD3zeta, or CD247), FcR gamma, FcR beta, CD3 gamma, CD3 delta, CD3 epsilon, CD5, CD22, CD79a, CD79b, and CD66d. A cytoplasmic signaling molecule in the CAR polypeptide can comprise a cytoplasmic signaling sequence of CD3 zeta or derived therefrom.

T cell persistence after activation is promoted by co-stimulatory molecules in addition to CD3 signaling. For this reason, the intercellular signaling portion of a CAR as described herein can further include one or more co-signaling domains from co-stimulatory proteins. The involvement of these co-signaling domains improve T cell proliferation, cytokine secretion, resistance to apoptosis, and in vivo persistence. In some embodiments of any of the aspects, co-signaling domains from a wide variety of co-stimulatory molecules can be used in the polypeptide as described herein, including CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. The co-stimulatory domains for a variety of TCR complex proteins are known in the art, e.g., as annotated in NCBI database entries for including CD27, CD28, 4-1BB (CD137), OX40, CD30, CD40, PD-1, ICOS, lymphocyte function-associated antigen-1 (LFA-1), CD2, CD7, LIGHT, NKG2C, and B7-H3. In several aspects, described herein are CAR polypeptides comprising at least one intracellular signaling portion.

In some embodiment of any of the aspects, the intracellular portion comprises the signaling domain of CD3-zeta and the signaling domain of CD28. In some embodiment of any of the aspects, the intracellular portion comprises the signaling domain of CD3-zeta and the signaling domain of 4-1BB. In some embodiments of any of the aspects, the intracellular domain comprises one or more co-signaling domains from a CD28 domain, a 4-1BB domain, a IL2Rα or IL2Rβ JAK3/STAT3 composite docking site, and a TGFβ-R SMAD2/3 docking site.

In one embodiment of any aspect, the intracellular signaling portion is a signaling domain from a protein selected from the group consisting of: TCRC; FcRy; FcRp; CD3zeta; CD3y; CD35; CD3s; CD3C; CD22; CD79a; CD79b; CD66d; CARD11; CD2; CD7; CD27; CD28; CD30; CD40; CD54 (ICAM); CD83; CD134 (OX40); CD137 (4-1BB); CD150 (SLAMF1); CD152 (CTLA4); CD223 (LAG3); CD270 (HVEM); CD273 (PD-L2); CD274 (PD-L1); CD278 (ICOS); DAP10; LAT; KD2C SLP76; TRIM; ZAP70; and 41BB.

In some embodiments of any of the aspects, a CAR polypeptide as described herein comprises the intracellular signaling domains of CD28 and CD3-zeta, which are encoded by a nucleic acid sequence presented as SEQ ID NO: 16 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 16 that maintains the same function or a codon-optimized version of SEQ ID NO: 16.

SEQ ID NO: 16 is a nucleic acid sequence that encodes a CAR intracellular signaling portion for CD28 and CD3-zeta:

| | |
|---|---|
| AGGAGTAAGA GGAGCAGGCT CCTGCACAGT GACTACATGA ACATGACTCC | 50 |
| CCGCCGCCCC GGGCCCACCC GCAAGCATTA CCAGCCCTAT GCCCCACCAC | 100 |
| GCGACTTCGC AGCCTATCGC TCCAGAGTGA AGTTCAGCAG GAGCGCAGAC | 150 |
| GCCCCCGCGT ACCAGCAGGG CCAGAACCAG CTCTATAACG AGCTCAATCT | 200 |
| AGGACGAAGA GAGGAGTACG ATGTTTTGGA CAAGAGACGT GGCCGGGACC | 250 |
| CTGAGATGGG GGGAAAGCCG AGAAGGAAGA ACCCTCAGGA AGGCCTGTAC | 300 |
| AATGAACTGC AGAAAGATAA GATGGCGGAG GCCTACAGTG AGATTGGGAT | 350 |
| GAAAGGCGAG CGCCGGAGGG GCAAGGGGCA CGATGGCCTT TACCAGGGTC | 400 |
| TCAGTACAGC CACCAAGGAC ACCTACGACG CCCTTCACAT GCAGGCCCTG | 450 |
| CCCCCTCGC | 459 |

In some embodiments of any of the aspects, a CAR polypeptide as described herein comprises the intracellular signaling portion of 4-1BB and CD3-zeta, which is encoded by a nucleic acid sequence comprising SEQ ID NO: 17 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 18 that maintains the same function or a codon-optimized version of SEQ ID NO: 17.

SEQ ID NO: 17 is a nucleic acid sequence that encodes a CAR intracellular signaling portion for 4-1BB and CD3-zeta:

| | |
|---|---|
| AAACGGGGCA GAAAGAAACT CCTGTATATA TTCAAACAAC CATTTATGAG | 50 |
| ACCAGTACAA ACTACTCAAG AGGAAGATGG CTGTAGCTGC CGATTTCCAG | 100 |
| AAGAAGAAGA AGGAGGATGT GAACTGAGAG TGAAGTTCAG CAGGAGCGCA | 150 |
| GACGCCCCCG CGTACCAGCA GGGCCAGAAC CAGCTCTATA ACGAGCTCAA | 200 |
| TCTAGGACGA AGAGAGGAGT ACGATGTTTT GGACAAGAGA CGTGGCCGGG | 250 |
| ACCCTGAGAT GGGGGGAAAG CCGAGAAGGA AGAACCCTCA GGAAGGCCTG | 300 |
| TACAATGAAC TGCAGAAAGA TAAGATGGCG GAGGCCTACA GTGAGATTGG | 350 |
| GATGAAAGGC GAGCGCCGGA GGGGCAAGGG GCACGATGGC CTTTACCAGG | 400 |
| GTCTCAGTAC AGCCACCAAG GACACCTACG ACGCCCTTCA CATGCAGGCC | 450 |
| CTGCCCCCTC GC | 462 |

In several aspects, described herein are CAR polypeptides comprising at least one detectable marker. As used herein, the term “detectable marker” refers to a moiety that, when attached to the CAR polypeptide, confers detectability upon that polypeptide or another molecule to which the polypeptide binds. In some embodiments of any of the aspects, the CAR polypeptide comprises one detectable marker and/or detectable polypeptide. In some embodiments of any of the aspects, the CAR polypeptide comprises a detectable marker or detectable polypeptide C-terminal of the intracellular signaling portion.

In some embodiments of any of the aspects, fluorescent moieties can be used as detectable markers, but detectable markers also include, for example, isotopes, fluorescent proteins and peptides, enzymes, components of a specific binding pair, chromophores, affinity tags as defined herein, antibodies, colloidal metals (i.e. gold) and quantum dots. Detectable markers can be either directly or indirectly detectable. Directly detectable markers do not require additional reagents or substrates in order to generate detectable signal. Examples include isotopes and fluorophores. Indirectly detectable markers require the presence or action of one or more co-factors or substrates. Examples include enzymes such as β-galactosidase which is detectable by generation of colored reaction products upon cleavage of substrates such as the chromogen X-gal (5-bromo-4-chloro-3-indoyl-β-D-galactopyranoside), horseradish peroxidase which is detectable by generation of a colored reaction product in the presence of the substrate diaminobenzidine and alkaline phosphatase which is detectable by generation of colored reaction product in the presence of nitroblue tetrazolium and 5-bromo-4-chloro-3-indolyl phosphate, and affinity tags. Non-limiting examples of affinity tags include Strep-tags, chitin binding proteins (CBP), maltose binding proteins (MBP), glutathione-S-transferase (GST), FLAG-tags, HA-tags, Myc-tags, poly(His)-tags as well as derivatives thereof. In some embodiments of any of the aspects, the detectable marker is selected from GFP, V5, HA1, Myc, VSV-G, HSV, FLAG, HIS, mCherry, AU1, and biotin.

In some embodiments of any of the aspects, the detectable marker can be located anywhere within a CAR polypeptide as described herein. In some embodiments of any of the aspects, the detectable marker is located between any portion of a CAR polypeptide as described herein, but is not found within a functional domain or portion or does not disrupt the function of a domain or portion. In some embodiments of any of the aspects, the detectable marker is located at the C-terminal end of the polypeptide. Such a marker can be used to detect the intracellular expression of the CAR polypeptide. In some embodiments of any of the aspects, the detectable marker located at the C-terminal end of the polypeptide comprises NeonGreen or another marker as described herein.

In some embodiments of any of the aspects, the detectable marker of a CAR polypeptide as described herein is encoded by a nucleic acid sequence comprising SEQ ID NOs: 18 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to one of SEQ ID NOs: 18 that maintains the same function or a codon-optimized version of one of SEQ ID NOs: 18.

SEQ ID NO: 18 is a nucleic acid sequence that encodes a CAR NeonGreen detectable marker:

| | |
|---|---|
| atggtcagca aaggcgaaga agacaacatg gcatcactgc cagcgacaca | 50 |
| cgaacttcat attttcggct ccatcaatgg tgtggacttc gatatggttg | 100 |
| gacagggtac tgggaaccct aacgatggat atgaggagtt gaacttgaag | 150 |
| agtaccaagg gcgatcttca gttttcacct tggattctcg tacctcatat | 200 |
| aggttatggc tttcatcaat atctgcccta tcctgacggt atgtcacctt | 250 |
| tccaagcagc tatggttgac gggagcggtt atcaggttca ccggacaatg | 300 |
| cagtttgaag atggagcctc tttgaccgta aattatcggt atacttatga | 350 |
| gggcagccat attaaaggcg aggctcaagt taaggggact gggttccccg | 400 |
| cggatggtcc cgtaatgaca aacagtctga cggctgcaga ctggtgccgc | 450 |
| tctaagaaga catatccaaa tgacaagacg attattagca ctttcaagtg | 500 |
| gtcatacact acaggaaatg gtaaacgcta taggagcact gcccggacca | 550 |
| cgtatacttt cgcgaagccg atggcggcaa attatttgaa aaaccaaccg | 600 |
| atgtatgtgt tcagaaagac agaactcaag cattctaaaa ctgaactgaa | 650 |
| cttcaaagag tggcagaaag cctttactga tgtaatgggc atggacgaac | 700 |
| tctataagta a | 711 |

In some embodiments of any of the aspects, CAR polypeptides as described herein, especially those that are administered to a subject or those that are part of a pharmaceutical composition, do not comprise detectable markers that are immunogenic. In some embodiments of any of the aspects, CAR polypeptides as described herein do not comprise GFP, mCherry, HA1, or any other immunogenic markers.

In some embodiments of any of the aspects, the CARs further comprise a cleavage site between the intracellular signal portion and the detectable marker. In some embodiments of any of the aspects, cleavage site is a short amino acid sequence (e.g., approximately 18-22 aa-long peptides) that can catalyze its own cleavage. In some embodiments of any of the aspects, the cleavage site belongs to the 2A peptide family. Non-limiting examples of 2A peptides include P2A, E2A, F2A and T2A. F2A is derived from foot-and-mouth disease virus 18; E2A is derived from equine rhinitis A virus; P2A is derived from porcine teschovirus-1 2A; T2A is derived from thosea asigna virus 2A. The cleavage site of these 2A peptides is known in the art. In some embodiments of any of the aspects, the N-terminus of the 2A peptide comprises the sequence “GSG” (Gly-Ser-Gly). In some embodiments of any of the aspects, the N-terminus of the 2A peptide does not comprise the sequence “GSG” (Gly-Ser-Gly).

The 2A-peptide-mediated cleavage commences after protein translation. The cleavage is triggered by breaking of peptide bond between the Proline (P) and Glycine (G) in the C-terminal of the 2A peptide. The molecular mechanism of 2A-peptide-mediated cleavage involves ribosomal “skipping” of glycyl-prolyl peptide bond formation rather than true proteolytic cleavage. Different 2A peptides have different efficiencies of self-cleaving, with P2A being the most efficient and F2A the least efficient. Therefore, up to 50% of F2A-linked proteins can remain in the cell as an uncleaved protein.

In some embodiments of any of the aspects, the cleavage site of a CAR as described herein is encoded by a nucleic acid sequence presented in SEQ ID NO: 19 or a nucleic acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to one of SEQ ID NOs: 19 that maintains the same function or a codon-optimized version of one of SEQ ID NOs: 19. In some embodiments of any of the aspects, the cleavage site comprises, consists of, or consists essentially of the polypeptide encoded by SEQ ID NO: 2 or a polypeptide that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the polypeptide encoded by SEQ ID NO: 2.

SEQ ID NO: 19 is a nucleic acid sequence that encodes a CAR cleavage site.

| | |
|---|---|
| ggatccggcg agggcagggg aagtcttcta acatgcgggg acgtggagga | 50 |
| aaatcccggg ccc | 63 |

In some embodiments of any of the aspects, the T cell or T cell population described herein comprises, consists of, or consists essentially of Regulatory T cells (Tregs). As used herein, “regulatory T cell” or “Treg” refers to those T cells (lymphocytes) that have immunoregulatory properties and the ability to suppress the proliferation and/or effector function of other T cell populations. A number of cell surface molecules are used to characterize and define Treg cells as described below herein. Regulatory T cells or Treg cells play an important role for the maintenance of immunological tolerance by suppressing the action of autoreactive effector cells and have been shown to be critically involved in preventing the development of autoimmune reactions.

In some embodiments of any of the aspects, a Treg cell can be a T cell expressing one or markers selected from the group consisting of CTLA4; PDL1; FOXP3; LAP; GARP; CD25; CD4; and CD27. In some embodiments of any of the aspects, a Treg cell can be a T cell expressing a detectable level of one or markers selected from the group consisting of CTLA4; PDL1; FOXP3; LAP; GARP; CD25; CD4; and CD27. In some embodiments of any of the aspects, a Treg cell can be a T cell positive for one or markers selected from the group consisting of CTLA4; PDL1; FOXP3; LAP; GARP; CD25; CD4; and CD27. In some embodiments of any of the aspects, a Treg cell can be a CD8− (e.g., NCBI Gene ID: 925) CD4+ (e.g., NCBI Gene ID: 920) CD3+ cell. In some embodiments of any of the aspects a Treg cell expresses Foxp3. In some embodiments of any of the aspects, a Treg cell expresses CD4 and CD25.

“X+”, wherein “X” is a cell surface marker, indicates the marker is present in the indicated cell, while “X−” indicates the marker is not present. One skilled in the art will be capable of assessing the molecules present on a cell using standard techniques, for example using immunofluorescence to detect commercially available antibodies bound to the marker molecules. Such designators are often used when sorting or identifying cells by FACS, in which gates can be established to divide cells based on the level of expression of the marker.

In one embodiment, the method for preparing the engineered cells described herein comprises obtaining a population of cells and enriching for the CD25+ T regulatory cells, for example by using antibodies specific to CD25. Methods for enriching CD25+ T regulatory cells from a population of cells will be apparent to a person of skill in the art and can comprise the use of methods such as FACS or affinity purification. In some embodiments, a population of Treg enriched cells comprises less than 30%, 20%, 10%, 5% or less non-Treg cells. In some embodiments, the vectors encoding the CARs described herein are transfected into Treg-enriched cells. Treg cells, e.g., Treg enriched cells, expressing a CAR may be used to induce tolerance to the antigen targeted by or specifically bound by the CAR.

CAR-T cell therapy uses T cells engineered with CARs for therapy. The premise of CAR-T immunotherapy is to modify T cells to recognize specific molecules or cells in order to more effectively target, block, or destroy them. T cells are harvested, genetically altered, then the resulting CAR-T cells are infused into patients to treat them. CAR-T cells can be derived from T cells in a patient's own blood (autologous) or derived from the T cells of another healthy donor (allogeneic). Once isolated, these T cells are genetically engineered to express a specific CAR, which programs them to target an antigen.

Further discussion of CARs can be found, e.g., in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

In one aspect of any of the embodiments, described herein is a nucleic acid molecule encoding a CAR described herein. In one aspect of any of the embodiments, described herein is a vector, e.g., an expression vector, comprising a nucleic acid molecule encoding a CAR described herein.

In some embodiments of any of the embodiments, a vector described herein is an expression vector or plasmid, e.g., a recombinant vector or plasmid. As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector; wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

In some embodiments of any of the aspects, the promoter is a eukaryotic or human constitutive promoter, including but not limited to a FoxP3 promoter, a Fox P3 and IKZF2/Helios hybrid promoter, a MND promoter, and the like. In some embodiments of any of the aspects, the vector comprises a human FoxP3 promoter (e.g., SEQ ID NO: 20) which is a constitutive promoter of human origin that can be used to drive gene expression in regulatory T cells in vitro and in vivo contexts.

In some embodiments of any of the aspects, the FoxP3 promoter of a CAR as described herein is encoded by a nucleic acid sequence presented in SEQ ID NO: 20 or a nucleic acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to SEQ ID NO: 20 that maintains the same function or a codon-optimized version of SEQ ID NO: 20.

SEQ ID NO: 20 is a nucleic acid sequence that encodes a FoxP3 promoter.

| | |
|---|---|
| tcgaaagctt ggcgcgccat atctaggtta gtcttttttt ctgtggcttc | 50 |
| tgtctctggt tttgtgctta gaaagtcctt tcctacttga aaatgagata | 100 |
| aatgttcacc tatgttggct tctagtctct tttatggctt cattttttcc | 150 |
| atttactata gaggttaaga gtgtgggtac tggagccaga ctgtctggga | 200 |
| caaacccagc gtcaccccaa gccctatgtg tgatttttag ccaggcactt | 250 |
| aacctctcca tccaagaagg gccaggtctt cagagctagg ggcttgtcat | 300 |
| agtggccaga tggacatcac ctaccacatc caccagcacc catgtcaccc | 350 |
| cacctgggcc aagcctgctg caggacaggg cagccagttc tcggaacgaa | 400 |
| acctgtgggg tggggtatct gccctcttct cttcctccgt ggtgtcgatg | 450 |
| aagcccggcg catccggccg ccatgacgtc aatggcggaa aaatctgggc | 500 |
| aagtcggggg ctgtgacaac agggcccaga tgcagacccc gatatgaaaa | 550 |
| cataatctgt gtcccagaaa catcccccat tcagcttctg agaaacccag | 600 |
| tcagaaaggg acgtcccaac agaggccctg ggcccaggat ggggcaggca | 650 |
| gggtggggta cctggaccta caggtgccga cctttactgt ggcactgggc | 700 |
| gggagggggg ctggctgggg cacaggaagt ggtttctggg tcccaggcaa | 750 |
| gtctgtgact tatgcagatg ttgcagggcc aagaaaatcc ccacctgcca | 800 |
| ggcctcagag attggaggct ctccccgacc tcccaatcct cccatccaca | 850 |
| catagagctt cagattctct ttctttcccc agagaccctc aaatatcctc | 900 |
| tcactcacag aatggtgtct ctgcctgcct cgggttggcc ctgtgattta | 950 |
| ttttagttct tttcccttgt tttttttttt tcaaactcta tacacttttg | 1000 |
| ttttaaaaac tgtggtttct catgagccct attatctcat tgatacctct | 1050 |
| cacctctgtg gtgaggggaa gaaatcatat tttcagatga ctcgtaaagg | 1100 |
| gcaaagaaaa aaacccaaaa tttcaaaatt tccgtttaag tctcataatc | 1150 |
| aagaaaagga gaaacacaga gagagagaaa aaaaaaacta tgagaacccc | 1200 |
| tccccacccc gtgattatca gcgcacacac tcatcgaaaa aaatttggat | 1250 |
| tattagaaga gagaggtctg cggcttccac accgtacagc gtggtttttc | 1300 |
| ttctcggtat aaaagcaaag ttgtttttga tacgtgacag tttcccacaa | 1350 |
| gccaggctga tccttttctg tcagtccact tcaccaagcc tgcccttgga | 1400 |
| caaggacccg tgacctcaag atcaaggaaa ggaggatgga cgaacagggg | 1450 |
| ccaaactggt gggaggcaga ggtggtgggg gcagggatga taggccctgg | 1500 |
| atgtgcccac agggaccaag aagtgaggtt tccactgtct tgcctgccag | 1550 |
| ggcccctgtt cccccgctgg cagccacccc ctcccccatc atatcctttg | 1600 |
| ccccaaggct gctcagaggg gccccggtcc tggccccagc ccccacctcc | 1650 |
| gccccagaca caccccccag tcgagccctg cagccaaaca gagccttcac | 1700 |
| aaccagccac acagagcctg cctcagctgc tcgcacagat tacttcaggg | 1750 |
| ctggaaaagt cacacagaca cacaaaatgt cacaatcctg tccctcactc | 1800 |
| aacacaaacc ccaaaacaca gagagcctgc ctcagtacac tcaaacaacc | 1850 |
| tcaaagctgc atcatcacac aatcacacac aagcacagcc ctgacaaccc | 1900 |
| acacacccca aggcacgcac ccacagccag cctcagggcc cacaggggca | 2000 |
| ctgtcaacac aggggtgtgc ccagaggcct acacagaagc agcgtcagta | 2050 |
| ccctcaggat ctgaggtccc aacacgtgct cgctcacaca cacggcctgt | 2100 |
| tagaattcac ctgtgtatct cacgcatatg cacacgcaca gccccccagt | 2150 |
| gggtctcttg agtcccgtgc agacacacac agccacacac actgccttgc | 2200 |
| caaaaatacc ccgtgtctcc cctgccactc acctcactcc cattccctga | 2250 |
| gccctgatcc atgcctcagc ttagactgca gaggaactac tcatttattt | 2300 |
| gggatccaag gcccccaacc cacagtaccg tccccgtcga cgagctcgta | 2500 |
| tc | 2502 |

In some embodiments of any of the aspects, the promoter is the promoter provided in Accession No. MK012431 e.g., nucleotides 4650-6041 and 7338-8213 of Accession No. MK012431, the version of the sequence available on Feb. 28, 2023.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector comprises a selectable marker, e.g., for selectively amplifying the vector in bacteria. Non-limiting examples of selectable marker genes for use in bacteria include antibiotic resistance genes conferring resistance to ampicillin, tetracycline and kanamycin. The tetracycline (tet) and ampicillin (amp) resistance marker genes can be obtained from any of a number of commercially available vectors including pBR322 (available from New England BioLabs, Beverly, Mass., cat. no. 303-3s). The tet coding sequence is contained within nucleotides 86-476; the amp gene is contained within nucleotides 3295-4155. The nucleotide sequence of the kanamycin (kan) gene is available from vector pACYC 177, from New England BioLabs, Cat no. 401-L, GenBank accession No. X06402.

In some embodiments of any of the aspects, one or more of the nucleic acids encoding a CAR as described herein can be integrated into the genome of the cell.

In some embodiments of any of the aspects, an immune cell (e.g., Treg) comprising a CAR polypeptide can be used to treat an inflammatory condition or autoimmune disease. In some embodiments, an immune cell (e.g., T cell) comprising a CAR polypeptide directed against an autoimmune disease-specific antigen can be used to treat an autoimmune disease. “Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Autoantigens, as used herein, are endogenous proteins or fragments thereof that elicit this pathogenic immune response. Autoantigen can be any substance or a portion thereof normally found within a mammal that, in an autoimmune disease, becomes the primary (or a primary) target of attack by the immune system. The term also includes antigenic substances that induce conditions having the characteristics of an autoimmune disease when administered to mammals. Additionally, the term includes peptic subclasses consisting essentially of immunodominant epitopes or immunodominant epitope regions of autoantigens. Immunodominant epitopes or regions in induced autoimmune conditions are fragments of an autoantigen that can be used instead of the entire autoantigen to induce the disease. In humans afflicted with an autoimmune disease, immunodominant epitopes or regions are fragments of antigens specific to the tissue or organ under autoimmune attack and recognized by a substantial percentage (e.g. a majority though not necessarily an absolute majority) of autoimmune attack T-cells.

Autoantigens that are known to be associated with autoimmune disease include myelin proteins with demyelinating diseases, e.g. multiple sclerosis and experimental autoimmune myelitis; collagens and rheumatoid arthritis; insulin, proinsulin, glutamic acid decarboxylase 65 (GAD65); islet cell antigen (ICA512; ICA12) with insulin dependent diabetes.

A common feature in a number of autoimmune related diseases and inflammatory conditions is the involvement of pro-inflammatory CD4+ T cells. These T cells are responsible for the release of inflammatory, Th1 type cytokines. Cytokines characterized as Th1 type include interleukin 2 (IL-2), γ-interferon, TNFα and IL-12. Such pro-inflammatory cytokines act to stimulate the immune response, in many cases resulting in the destruction of autologous tissue. Cytokines associated with suppression of T cell response are the Th2 type, and include IL-10, IL-4 and TGF-β. It has been found that Th1 and Th2 type T cells may use the identical antigen receptor in response to an immunogen; in the former producing a stimulatory response and, in the latter, a suppressive response.

Described herein is a method of treating an autoimmune disease, which comprises administering an effective amount of a CAR or CAR-T composition to a patient in need thereof. In some embodiments of any one of the methods described, the autoimmune disorder is selected from the group consisting of thyroiditis, type 1 diabetes mellitus, Hashimoto's thyroidits, Graves' disease, celiac disease, multiple sclerosis, Guillain-Barre syndrome, Addison's disease, and Raynaud's phenomenon, Goodpasture's disease, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, an immune complex disorder such as immune complex nephritis, antibody-mediated nephritis, polyneuropathies, chronic neuropathy such as IgM polyneuropathies or IgM-mediated neuropathy, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, idiopathic hypothyroidism, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, lupoid hepatitis, giant-cell hepatitis, autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies including channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, Sampter's syndrome, Caplan's syndrome, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, cyclitis such as chronic cyclitis, heterochronic cyclitis, iridocyclitis (acute or chronic), or Fuch's cyclitis, Henoch-Schonlein purpura, SCID, sepsis, endotoxemia, post-vaccination syndromes, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, keratoconjunctivitis sicca, idiopathic nephritic syndrome, minimal change nephropathy, benign familial and ischemia-reperfusion injury, transplant organ reperfusion, retinal autoimmunity, aphthae, aphthous stomatitis, arteriosclerotic disorders, aspermiogenesis, autoimmune hemolysis, Boeck's disease, enteritis allergica, erythema nodosum leprosum, idiopathic facial paralysis, chronic fatigue syndrome, febris rheumatica, Hamman-Rich's disease, sensoneural hearing loss, ileitis regionalis, leucopenia, transverse myelitis, primary idiopathic myxedema, ophthalmia symphatica, polyradiculitis acuta, pyoderma gangrenosum, acquired splenic atrophy, vitiligo, toxic-shock syndrome, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndrome type I, adult-onset idiopathic hypoparathyroidism (AOIH), myocarditis, nephrotic syndrome, primary sclerosing cholangitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, granulomas containing eosinophils, seronegative spondyloarthritides, polyendocrine autoimmune disease, sclerosing cholangitis, sclera, episclera, Bruton's syndrome, transient hypogammaglobulinemia of infancy, Wiskott-Aldrich syndrome, ataxia telangiectasia syndrome, angiectasis, autoimmune disorders associated with collagen disease, rheumatism, allergic hypersensitivity disorders, glomerulonephritides, reperfusion injury, ischemic re-perfusion disorder, lymphomatous tracheobronchitis, inflammatory dermatoses, dermatoses with acute inflammatory components, and autoimmune uveoretinitis (AUR).

Described herein is a method of treating an autoimmune or inflammatory condition, which comprises administering an effective amount of a CAR or CAR-T composition to a patient in need thereof. In some embodiments of any one of the methods described, the autoimmune or inflammatory condition is selected from the group consisting of an allograft rejection, a xenograft rejection, and graft-vs. host disease.

In some embodiments, the methods described herein relate to the treatment or prevention of transplant rejection or GVHD in a subject having a tissue transplant, organ transplant, or a hematopoietic stem cell transplant (HCT) with one or more compositions, CARs, or cells as described herein. In some embodiments of any of the aspects, the transplant rejection is an allograft rejection or a xenograft rejection. In another embodiment, the methods described herein relate to the treatment or prevention of Graft-versus-host disease (GVHD) in a subject having a tissue transplant, organ transplant, or an HCT with one or more compositions, CARs, or cells as described herein.

As used herein, “GVHD” refers to a disease characterized by the active process of donor cells attacking the recipient's own cells. GVHD can develop soon after a transplant, e.g., within weeks or months (acute GVHD), or can occur much later after the transplant, e.g., at least 3-6 months later (chronic GVHD). Symptoms of acute GVHD include, but are not limited to, skin rash or blisters, abdominal pain or discomfort, diarrhea, jaundice, and edema. Symptoms of chronic GVHD include, but are not limited to, changes to skin or nail texture, hair loss or thinning, muscle pain or weakness, blurred vision, mouth sores, shortness of breath, persistent cough, abdominal pain or discomfort, and diarrhea.

A subject can be identified as having or be at risk of having GVHD by a skilled clinician. Diagnostic tests useful in identifying a subject having GVHD are known in the art and will vary based on the type of transplant the subject has received. The diagnosis of GVHD is made by, for example, physical examination for the signs and symptoms for GVHD known in the art, serologic testing for dysfunction of the liver, gall bladder, kidney, and hematopoietic cells, histologic analysis of biopsies obtained from affected organs, and radiologic imaging of affected organs. In one embodiment, the method further comprises administering at least a second therapeutic. In one embodiment, the composition, CARs, or cells described herein are administered in combination with Abatacept (Orencia®) or Belatacept (Nulojix®). Abatacept and Belatacept, developed by Bristol-Meyers Squibb, are fusion proteins composed of the Fc region of the immunoglobulin IgG1 fused to the extracellular domain of CTLA-4. Abatacept is currently approved by the FDA for treatment of rheumatoid arthritis. Belatacept, which only differs from Abatacept by two amino acids, is an immunosuppressant intended to prevent rejection following a kidney transplant.

In some embodiments of any of the aspects, the transplant is vascularized composite allotransplantation (VCA). In another embodiment, the transplant is any type of transplants procedures, e.g., any heart transplant, any lung transplant, any liver transplant, any pancreas transplant, any cornea transplant, any trachea transplant, any kidney transplant, any skin transplant, or any autograft (e.g., a transplantation of tissue). In one embodiment of any of the aspects, the transplant is any allograft or xenograft. A skilled practitioner will be able to perform a transplant or identify a subject having had a transplant using standard procedural protocols.

The compositions and methods described herein can be administered to a subject to treat or prevent an autoimmune disease, inflammatory condition, or transplant rejection. In some embodiments, the methods described herein comprise administering an effective amount of compositions, CARs, or cells described herein to a subject in order to alleviate a symptom of an inflammatory condition, autoimmune disease or transplant rejection. As used herein, “alleviating a symptom of an inflammatory condition, autoimmune disease or transplant rejection” is ameliorating any condition or symptom associated with the inflammatory condition, autoimmune disease or transplant rejection. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

It can generally be stated that a pharmaceutical composition comprising the cells, e.g., T cells or CAR cells, described herein may be administered at a dosage of 1 to 10¹⁰ cells/kg body weight, preferably 10³ to 10⁸ cells/kg body weight, including all integer values within those ranges. The number of cells will depend upon the ultimate use for which the composition is intended as will the type of cells included therein. For uses described herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen, lower numbers of cells, in the range of 10⁶/kilogram (10⁶-10¹¹ per patient) may be administered. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as described herein to enhance induction of the immune response. In some embodiments, the dosage can be from about 1×10² cells to about 1×10¹⁰ cells per kg of body weight. In some embodiments, the dosage can be from about 1×10⁵ cells to about 1×10⁸ cells per kg of body weight. In some embodiments, the dosage can be from about 1×10⁶ cells to about 1×10⁷ cells per kg of body weight. In some embodiments, the dosage can be about 1×10⁶ cells per kg of body weight. In some embodiments, one dose of cells can be administered. In some embodiments, the dose of cells can be repeated, e.g., once, twice, or more. In some embodiments, the dose of cells can be administered on, e.g., a daily, weekly, or monthly basis.

The dosage ranges for the agent, e.g., a CAR, cell, or composition described herein depend upon the potency, and encompass amounts large enough to produce the desired effect e.g., prevention of transplant rejection, reduction in inflammation, etc. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 0.5 mg/kg body weight. In some embodiments, the dose range is from 5 μg/kg body weight to 100 μg/kg body weight. Alternatively, the dose range can be titrated to maintain serum levels between 1 g/mL and 1000 g/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

Administration of the doses recited above can be repeated. In some embodiments, the doses are given once a day, or multiple times a day, for example but not limited to three times a day. In some embodiments, the doses recited above are administered daily for several weeks or months. The duration of treatment depends upon the subject's clinical progress and responsiveness to therapy.

In some embodiments, the dose can be from about 2 mg/kg to about 15 mg/kg. In some embodiments, the dose can be about 2 mg/kg. In some embodiments, the dose can be about 4 mg/kg. In some embodiments, the dose can be about 5 mg/kg. In some embodiments, the dose can be about 6 mg/kg. In some embodiments, the dose can be about 8 mg/kg. In some embodiments, the dose can be about 10 mg/kg. In some embodiments, the dose can be about 15 mg/kg. In some embodiments, the dose can be from about 100 mg/m² to about 700 mg/m². In some embodiments, the dose can be about 250 mg/m². In some embodiments, the dose can be about 375 mg/m². In some embodiments, the dose can be about 400 mg/m². In some embodiments, the dose can be about 500 mg/m².

In some embodiments, the dose can be administered intravenously. In some embodiments, the intravenous administration can be an infusion occurring over a period of from about 10 minutes to about 3 hours. In some embodiments, the intravenous administration can be an infusion occurring over a period of from about 30 minutes to about 90 minutes.

In some embodiments the dose can be administered about weekly. In some embodiments, the dose can be administered weekly. In some embodiments, the dose can be administered weekly for from about 12 weeks to about 18 weeks. In some embodiments the dose can be administered about every 2 weeks. In some embodiments the dose can be administered about every 3 weeks. In some embodiments, the dose can be from about 2 mg/kg to about 15 mg/kg administered about every 2 weeks. In some embodiments, the dose can be from about 2 mg/kg to about 15 mg/kg administered about every 3 weeks. In some embodiments, the dose can be from about 2 mg/kg to about 15 mg/kg administered intravenously about every 2 weeks. In some embodiments, the dose can be from about 2 mg/kg to about 15 mg/kg administered intravenously about every 3 weeks. In some embodiments, the dose can be from about 200 mg/m2 to about 400 mg/m2 administered intravenously about every week. In some embodiments, the dose can be from about 200 mg/m2 to about 400 mg/m2 administered intravenously about every 2 weeks. In some embodiments, the dose can be from about 200 mg/m2 to about 400 mg/m2 administered intravenously about every 3 weeks. In some embodiments, a total of from about 2 to about 10 doses are administered. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, the administration occurs for a total of from about 4 weeks to about 12 weeks. In some embodiments, the administration occurs for a total of about 6 weeks. In some embodiments, the administration occurs for a total of about 8 weeks. In some embodiments, the administration occurs for a total of about 12 weeks. In some embodiments, the initial dose can be from about 1.5 to about 2.5-fold greater than subsequent doses.

In some embodiments, the dose can be from about 1 mg to about 2000 mg. In some embodiments, the dose can be about 3 mg. In some embodiments, the dose can be about 10 mg. In some embodiments, the dose can be about 30 mg. In some embodiments, the dose can be about 1000 mg. In some embodiments, the dose can be about 2000 mg. In some embodiments, the dose can be about 3 mg given by intravenous infusion daily. In some embodiments, the dose can be about 10 mg given by intravenous infusion daily. In some embodiments, the dose can be about 30 mg given by intravenous infusion three times per week.

A therapeutically effective amount is an amount of an agent that is sufficient to produce a statistically significant, measurable change in, or prevent the occurrence of an inflammatory condition, autoimmune disease, transplant rejection, or GVHD. Such effective amounts can be gauged in clinical trials as well as animal studies.

An agent can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. It is preferred that the compounds used herein are administered orally, intravenously or intramuscularly. Local administration, e.g., directly to the site of an organ or tissue transplant is also specifically contemplated.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

In some embodiments, the methods further comprise administering a composition, CAR, or cell described herein along with one or more additional inflammatory, autoimmune, GVHD, or transplant rejection agents, biologics, drugs, or treatments as part of a combinatorial therapy. Exemplary treatments for transplant rejection or GVHD include but are not limited to, Immunosuppressive drugs, e.g., Cyclosporine (Neoral, Sandimmune, Gengraf, and Restasis), Tacrolimus (Prograf, Protopic, Astagraf XL, and Envarsus XR), Methotrexate (Trexall, Rasuvo, Rheumatrex, and Otrexup (PF)), Sirolimus (Rapamune), Mycophenolic acid (Myfortic and CellCept), Rituximab (Rituxan), etanercept (Enbrel), pentostatin (Nipent), ruxolitinib (Jakafi); Chemotherapies, e.g., Methotrexate (Trexall, Rasuvo, Rheumatrex, and Otrexup (PF)), antithymocyte globulin (Atgam, Thymoglobulin); Steroids, e.g., Prednisone (Deltasone, Rayos, and Prednisone Intensol), Methylprednisolone (Medrol, Solu-Medrol, and Depo-Medrol), budesonide (Entocort EC, Uceris); Antifungal, e.g., Posaconazole (Noxafil); Antiviral drugs, e.g., Acyclovir (Zovirax and Sitavig), Valacyclovir (Valtrex); and Antibiotics, e.g., Sulfamethoxazole/Trimethoprim (Bactrim, Sulfatrim, and Bactrim DS); Protease inhibitors, e.g. alpha1-proteinase inhibitor (Zemaira); extracorporeal photopheresis; monoclonal antibodies (daclizumab (Zinbryta), basiliximab (Simulect)), Brentuximab vedotin (Adcetris), Alemtuzumab (Campath, Lemtrada), Tocilizumab (Actemra); infusion of mesenchymal stromal cells. Exemplary treatments for autoimmune disease include but are not limited to, Insulin, e.g., Insulin glulisine (Apidra and Apidra SoloStar), Insulin detemir (Levemir and Levemir FlexTouch), Insulin aspart (NovoLog, Novolog Flexpen, and Novolog PenFill), Insulin lispro (Humalog and Humalog KwikPen), Insulin, Insulin glargine (Lantus, Lantus Solostar, and Toujeo SoloStar); Dietary supplement, e.g., glucose tablets; and Hormones, e.g., Glucagon (GlucaGen and Glucagon Emergency Kit (human)), antidiabetic agents (Metformin (D-Care DM2, Fortamet, Glucophage, Glucophage XR, Glumetza, Riomet), glucagon-like peptide-1 (GLP-1) receptor agonist (liraglutide (Saxenda; Victoza) or semaglutide (Ozempic) or sodium-glucose co-transporter 2 (SGLT2) inhibitor: empagliflozin (Jardiance), canagliflozin (Invokana); sulfonylureas: glipizide (GlipiZIDE XL, Glucotrol, Glucotrol XL); Meglitinide Analogs: repaglinide (Prandin); Thiazolidinedione: pioglitazone (Actos); dipeptidyl peptidase-4 [DPP-4] inhibitors: Sitagliptin (Januvia), Saxagliptin (Onglyza), Linagliptin (Tradjenta), Alogliptin (Nesina)

The efficacy of a given treatment, e.g., for an inflammatory condition, autoimmune disease, transplant reject, or GVHD, can be determined by the skilled clinician. However, a treatment is considered “effective treatment,” as the term is used herein, if any one or all of the signs or symptoms of are altered in a beneficial manner or other clinically accepted symptoms are improved, or even ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

An effective amount for the treatment of a disease means that amount which, when administered to a mammal in need thereof, is sufficient to result in effective treatment as that term is defined herein, for that disease. Efficacy of an agent can be determined by assessing physical indicators of, for example autoimmune disease (e.g., result of an ANA), transplant rejection (e.g., high fever, tenderness at transplant site, etc.), or GVHD (e.g., redness, pain, or other symptoms at transplant site). Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay to assess reaction following transplant, level of inflammation, ANA measurement, among others. The dosage can be determined by a physician and adjusted, as necessary, to suit observed effects of the treatment.

Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. a reduction of inflammation, etc.). It is well within the ability of one skilled in the art to monitor efficacy of administration and/or treatment by measuring any one of such parameters, or any combination of parameters. Efficacy can be assessed in animal models of a condition described herein, for example treatment of an inflammatory condition, autoimmune disease, transplant rejection or GVHD. When using an experimental animal model, efficacy of treatment is evidenced when a statistically significant change in a marker is observed, e.g. inflammation.

In some embodiments, the technology described herein relates to a pharmaceutical composition comprising a CAR, as described herein, and optionally a pharmaceutically acceptable carrier. In some embodiments, the active ingredients of the pharmaceutical composition comprise a CAR as described herein. In some embodiments, the active ingredients of the pharmaceutical composition consist essentially of a CAR as described herein. In some embodiments, the active ingredients of the pharmaceutical composition consist of a CAR as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent as described herein.

In some embodiments, the pharmaceutical composition comprising a CAR as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a CAR as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an active ingredient can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled release of one or more active ingredients using, for example, hydroxypropylmethyl cellulose, other polymer matrices, gels, permeable membranes, osmotic systems (such as OROS® (Alza Corporation, Mountain View, Calif USA)), or a combination thereof to provide the desired release profile in varying proportions.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The term “improve” or “improvement,” when applied to a score in a standardized scale or rating, e.g., for disease symptoms or severity, means a statistically significant, favorable change in the scale or rating on that scale.

As used herein, a “reference level” refers to the level or value for a given parameter against which one compares the level or value in a given sample or situation to determine whether the level or value has changed in a meaningful way. A reference level can be a level in or from a sample that is not treated to change the parameter. A reference level can alternatively be a level in or from a normal or otherwise unaffected sample. A reference level can alternatively be a level in or from a sample obtained from a subject at a prior time point, for example, prior to a given treatment.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of inflammatory condition, autoimmune disease, transplant rejection, or GVHD. A subject can be male or female.

A subject can be one who has been previously diagnosed with or identified as suffering from or having a condition in need of treatment (e.g. an inflammatory condition, an autoimmune disease, transplant rejection, or GVHD) or one or more complications related to such a condition, and optionally, have already undergone treatment for an inflammatory condition, an autoimmune disease, transplant rejection, or GVHD or the one or more complications related to an inflammatory condition, autoimmune disease, transplant rejection, or GVHD. Alternatively, a subject can also be one who has not been previously diagnosed as having an inflammatory condition, autoimmune disease, transplant rejection, or GVHD or one or more complications related to an inflammatory condition, autoimmune disease, transplant rejection, or GVHD. For example, a subject can be one who exhibits one or more risk factors for an inflammatory condition, autoimmune disease, transplant rejection, or GVHD or one or more complications related to an inflammatory condition, autoimmune disease, transplant rejection, or GVHD or a subject who does not exhibit risk factors.

A “subject in need” of treatment for a particular condition can be a subject having that condition, diagnosed as having that condition, or at risk of developing that condition, e.g., an inflammatory condition, autoimmune disease, transplant rejection, or GVHD.

As used herein, an “appropriate control” refers to an untreated, otherwise identical cell or population (e.g., a subject who was not administered an agent described herein, or was administered by only a subset of agents described herein, as compared to a non-control cell).

In some embodiments, a nucleic acid encoding a CAR as described herein is comprised by a vector. In some of the aspects described herein, a nucleic acid sequence encoding CAR as described herein, or any module thereof, is operably linked to another sequence, e.g., a promoter in a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence that is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a CAR described herein, in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a nontarget. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. an inflammatory response, autoimmune disease, transplant rejection, or GVHD. The term “treating” includes reducing or alleviating at least one adverse effect or symptom of a condition associated with a disease or disorder, e.g. an inflammatory response, autoimmune disease, transplant rejection, or GVHD. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of an agent, e.g., a CAR, composition, or cell as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A chimeric antigen receptor (CAR) polypeptide comprising, from N-terminus to C-terminus:
   a) an extracellular recognition portion that specifically binds to OX40L;
   b) a transmembrane portion; and
   c) an intracellular signaling portion.
2. The CAR of paragraph 1, further comprising, C-terminal of the intracellular signaling portion, a detectable polypeptide.
3. The CAR of paragraph 2, wherein the detectable polypeptide is a fluorescent polypeptide.
4. The CAR of paragraph 3, wherein the fluorescent polypeptide is NeonGreen.
5. The CAR of any one of paragraph 2-4, further comprising a cleavage site between the intracellular signaling portion and the detectable polypeptide.
6. The CAR of paragraph 5, wherein the cleavage site is a cleavable T2A site or a tandem P2A-T2A site.
7. The CAR of any one of the preceding paragraphs, wherein the recognition portion is an antibody reagent or ligand functional domain.
8. The CAR of paragraph 7, wherein the antibody reagent is a scFV.
9. The CAR of any one of paragraph 7-8, wherein the antibody reagent is an anti-OX40L antibody reagent.
10. The CAR of paragraph 9, wherein the antibody reagent comprises CDR sequences at least 80% identical, at least 85% identical, at least 90% identical, at least 95% identical, or 100% identical to the six CDRs of SEQ ID NOs: 1-6.
11. The CAR of paragraph 9, wherein the antibody reagent comprises a sequence at least 80% identical, at least 85% identical, at least 90% identical, at least 95% identical, or 100% identical to the amino acid sequences of SEQ ID NO 7-12.
12. The CAR of any one of paragraph 1-11, wherein the intracellular signaling portion comprises one or more of: a CD28 co-signaling domain, a 41BB co-signaling domain, a IL2Rα JAK3 and IL2Rβ STAT5 composite docking site, a TGFβ-R SMAD2/3 docking site, and a CD3zeta signaling domain.
13. The CAR of any one of paragraph 1-11, wherein the intracellular signaling portion comprises a CD28 co-signaling domain and a CD3zeta domain.
14. The CAR of any one of paragraph 1-11, wherein the intracellular signaling portion comprises a 41BB co-signaling domain and a CD3zeta domain.
15. A nucleic acid molecule encoding the CAR of any of the preceding claims.
16. The nucleic acid molecule of paragraph 15, wherein the expression of the CAR is controlled by a Treg-specific promoter or a MND promoter.
17. The nucleic acid molecule of paragraph 16, wherein the Treg-specific promoter comprises a FoxP3 promoter or a FoxP3 and IKZF2/Helios hybrid promoter.
18. A vector comprising the nucleic acid molecule of any of paragraph 15-17.
19. A cell comprising the CAR of any one of paragraph 1-14, or the nucleic acid molecule or vector of any one of claims 15-18.
20. The cell of paragraph 19, wherein the cell is a Treg.
21. The cell of paragraph 20, wherein the Treg expresses Foxp3.
22. The cell of paragraph 20, wherein the Treg expresses CD4 and CD25.
23. A population of cells, at least 80% of which are cells according to paragraph 19.
24. A method of treating an autoimmune or inflammatory condition in a subject in need thereof, the method comprising administering to the subject a cell or population of cells of any of paragraph 19-23.
25. The method of paragraph 24, wherein said autoimmune or inflammatory condition comprises an allograft or xenograft rejection, or graft-vs. host disease (GVHD).
26. The method of paragraph 24, wherein said autoimmune or inflammatory condition is selected from the group consisting of: an inflammatory bowel disease; rheumatoid arthritis; type I diabetes mellitus or autoimmune insulitis; multiple sclerosis; autoimmune thyroiditis; autoimmune gastritis; autoimmune uveitis or uveoretinitis; autoimmune orchitis; autoimmune oophoritis; psoriasis; vitiligo; autoimmune prostatitis; any undesired immune response; tissue rejection; and an inflammatory condition.
27. The method of paragraph 24-26, wherein the population of Tregs are autologous to the subject.
28. The method of paragraph 24-26, wherein the population of Tregs are allogenic to the subject.
29. A cell or population of cells of any of paragraph 19-23, for use in a method of treating an autoimmune or inflammatory condition in a subject in need thereof, the method comprising administering the cell or population of cells to the subject.
30. The cell or population of cells of paragraph 24, wherein said autoimmune or inflammatory condition comprises an allograft or xenograft rejection, or graft-vs. host disease (GVHD).
31. The cell or population of cells of paragraph 24, wherein said autoimmune or inflammatory condition is selected from the group consisting of: an inflammatory bowel disease; rheumatoid arthritis; type I diabetes mellitus or autoimmune insulitis; multiple sclerosis; autoimmune thyroiditis; autoimmune gastritis; autoimmune uveitis or uveoretinitis; autoimmune orchitis; autoimmune oophoritis; psoriasis; vitiligo; autoimmune prostatitis; any undesired immune response; tissue rejection; and an inflammatory condition.
32. The cell or population of cells of paragraph 24-26, wherein the population of Tregs are autologous to the subject.
33. The cell or population of cells of paragraph 24-26, wherein the population of Tregs are allogenic to the subject.

### EXAMPLES

### Example 1

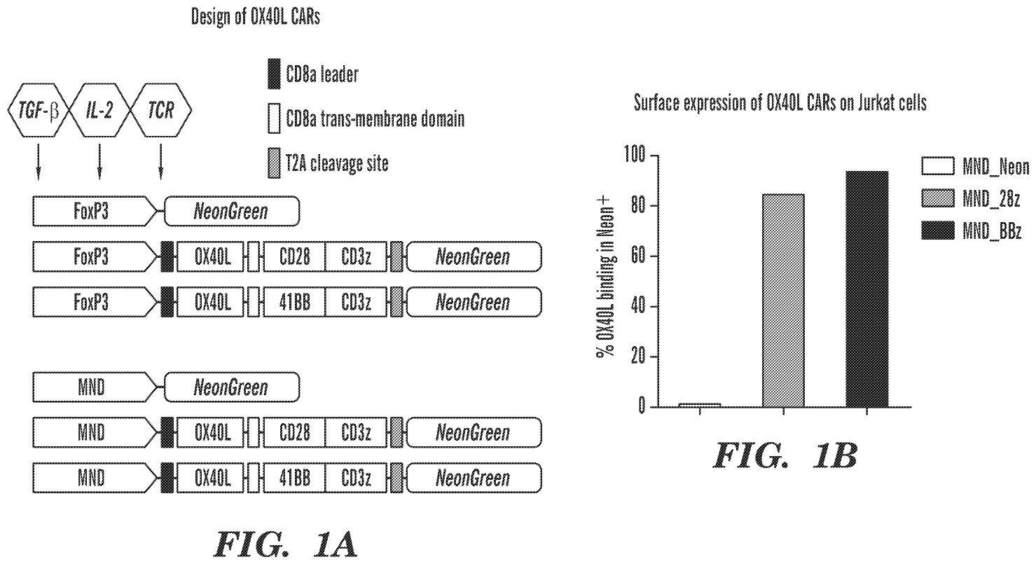

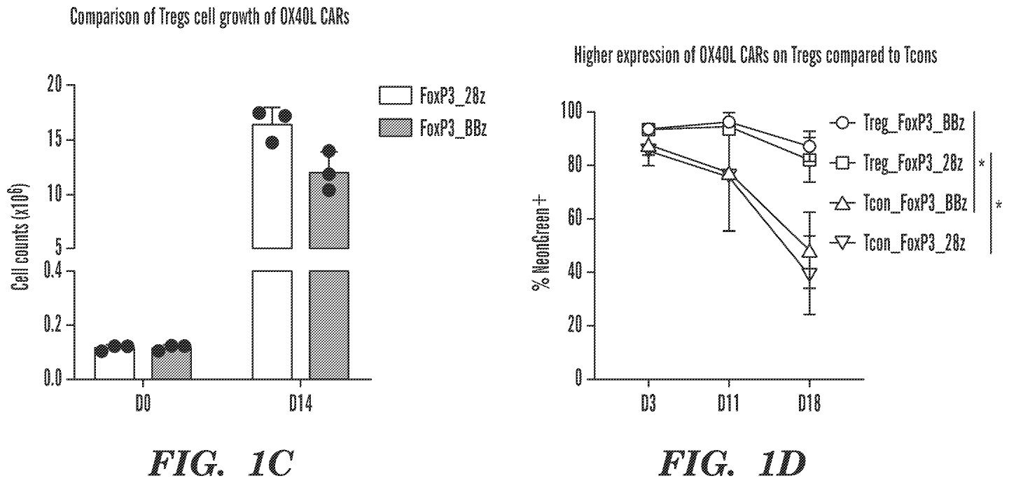

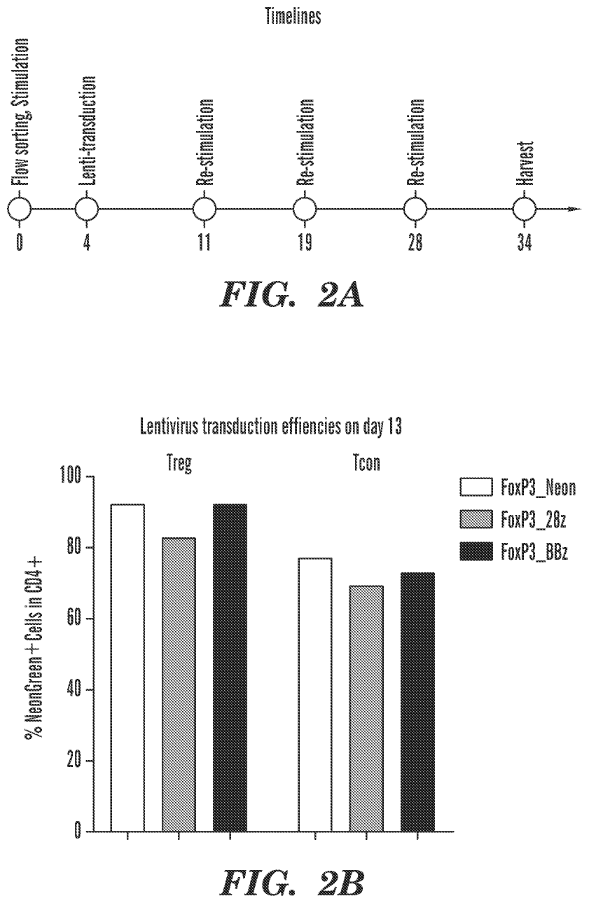

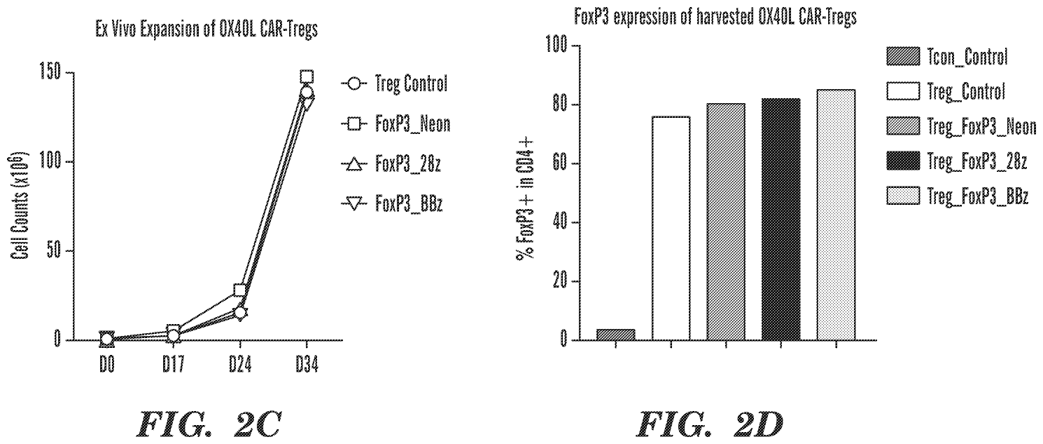

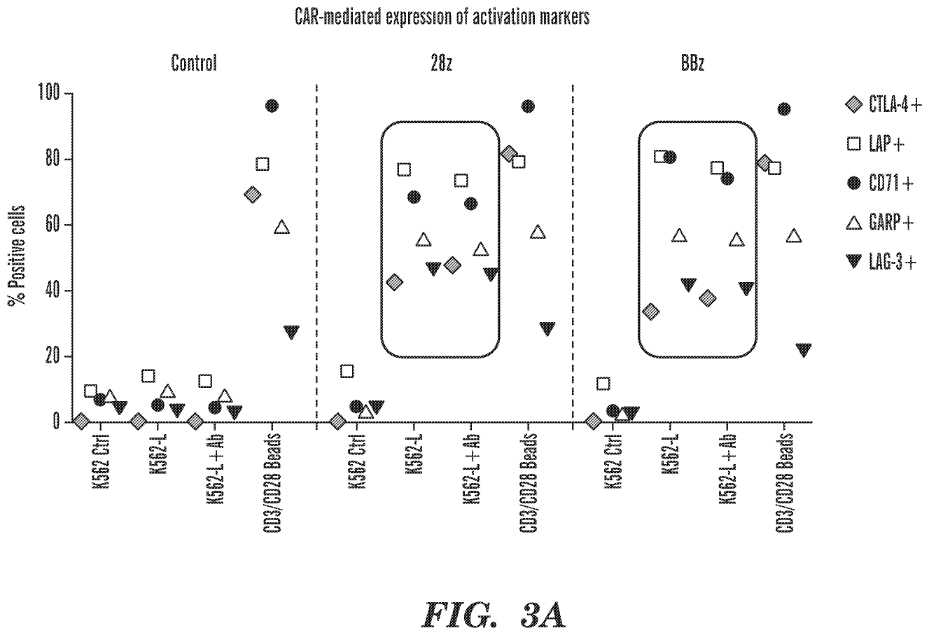

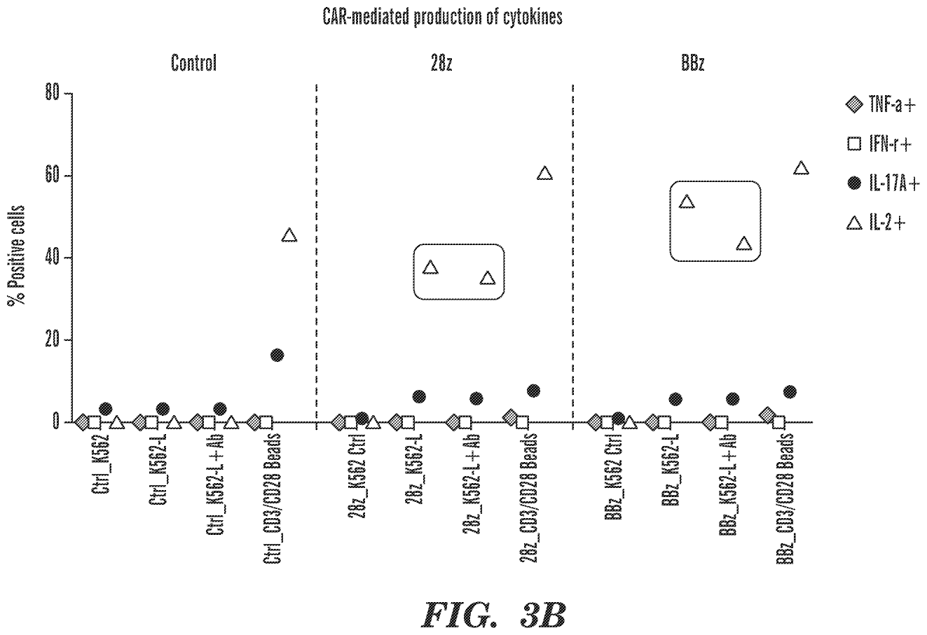

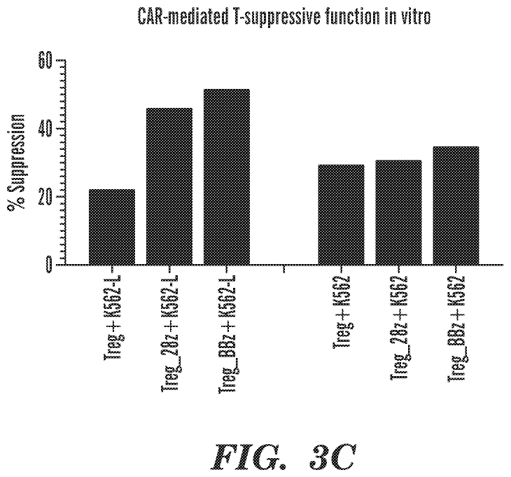

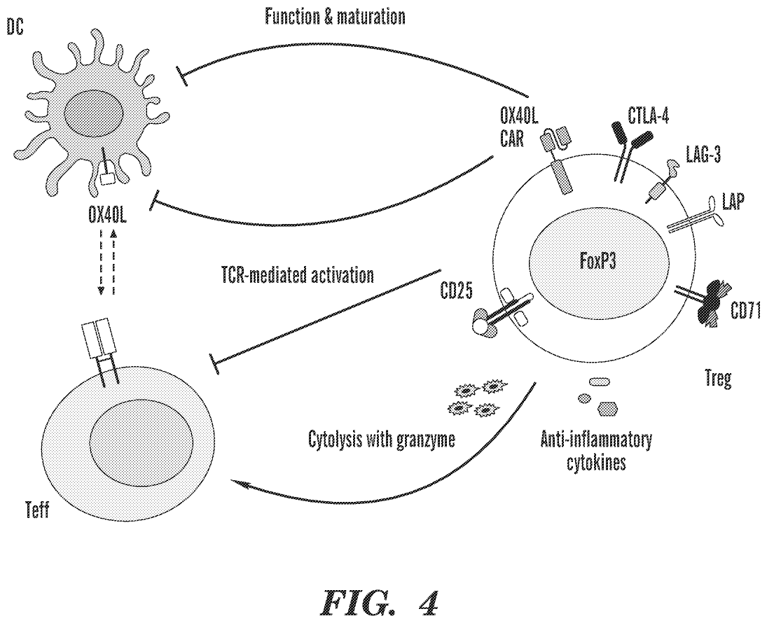

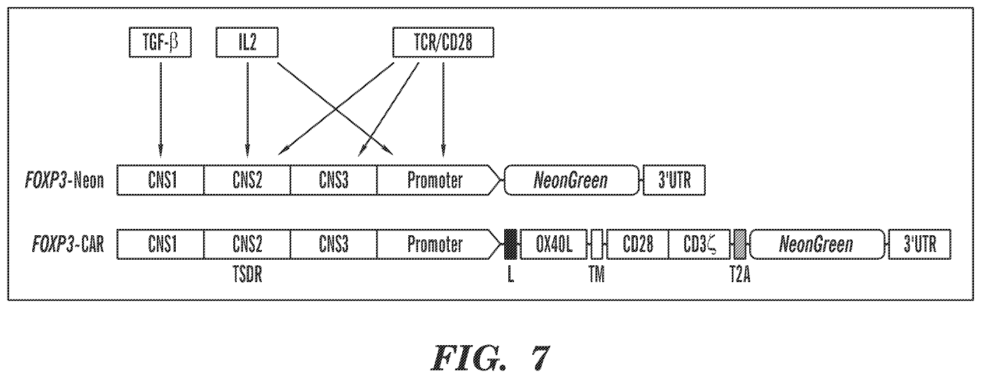

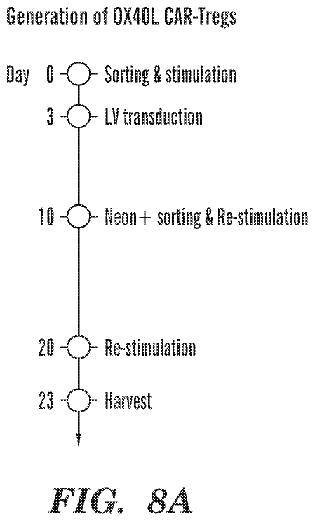

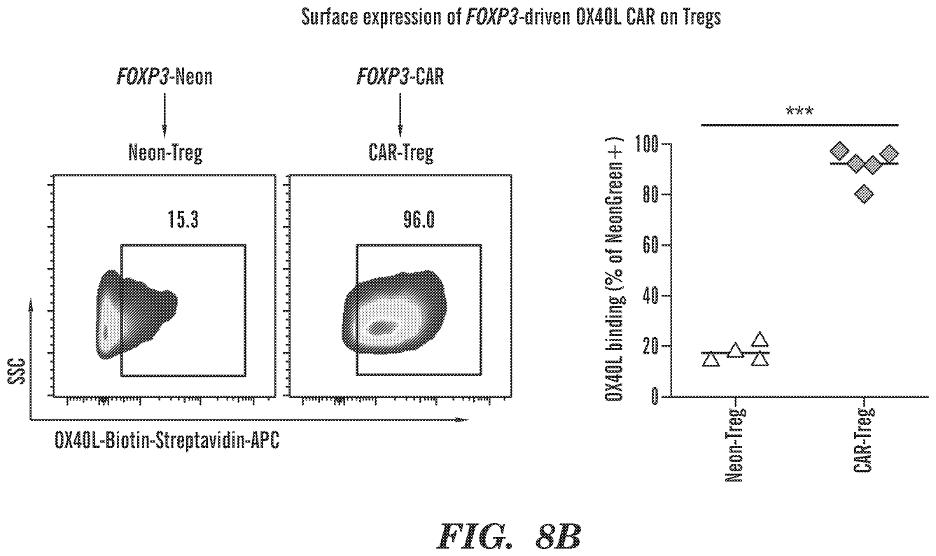

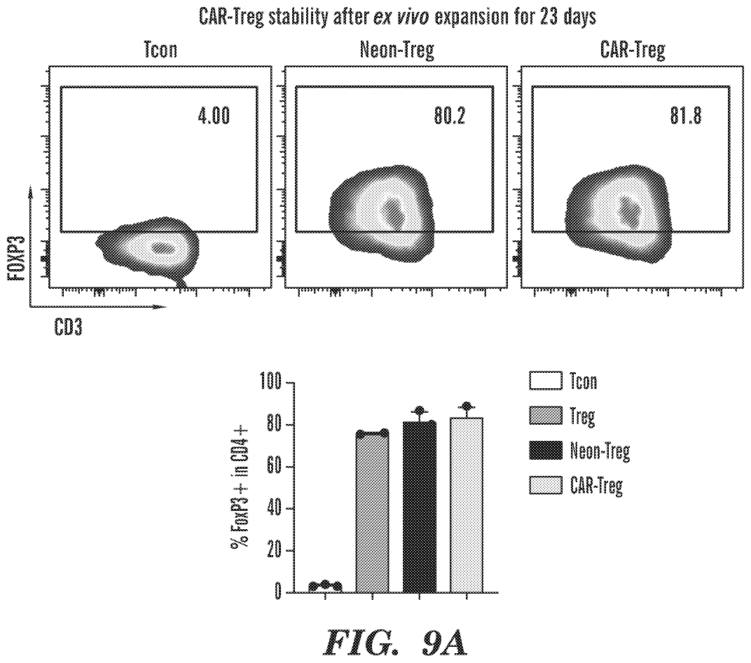

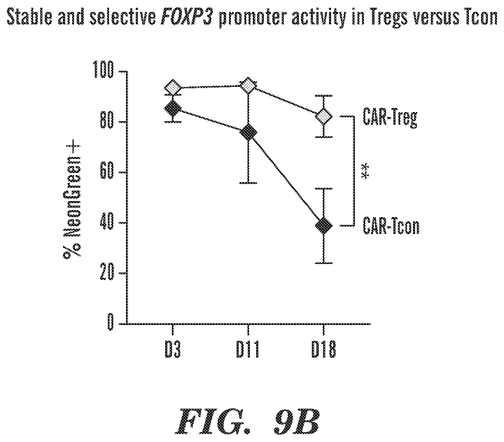

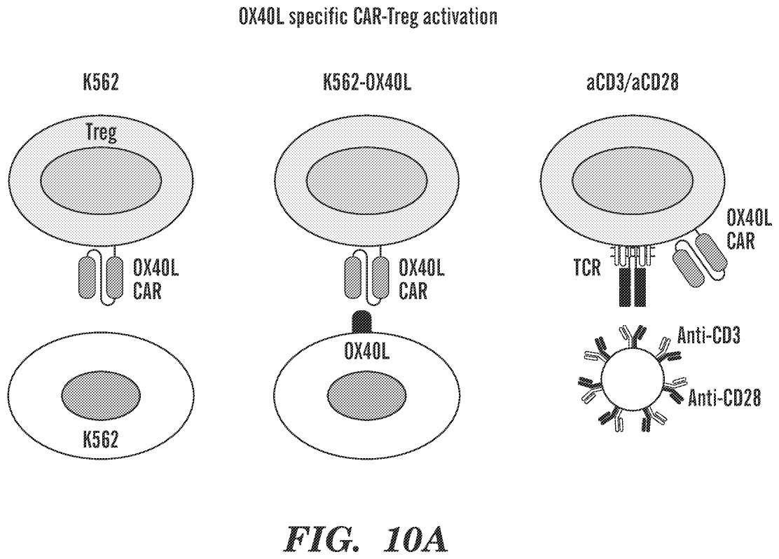

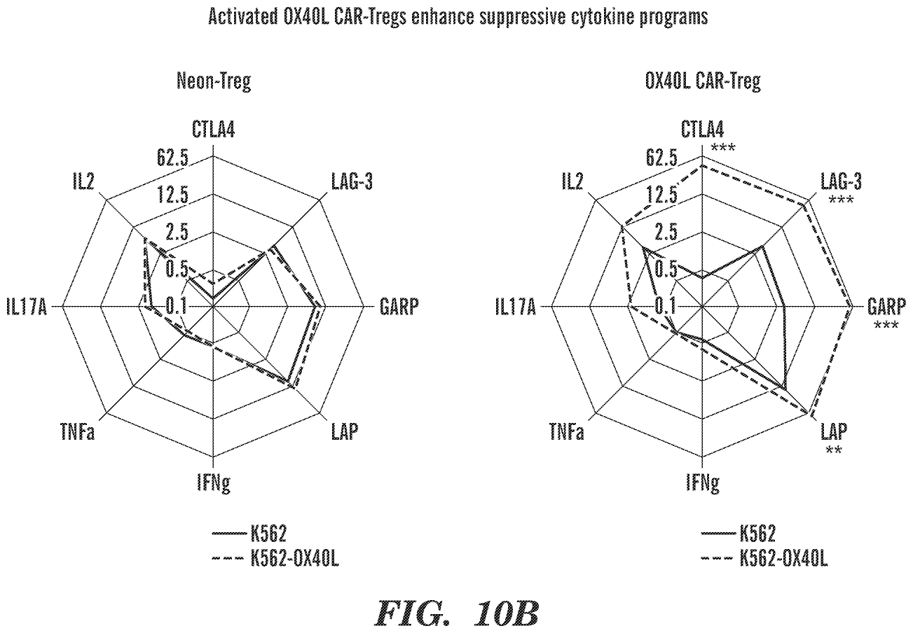

The inventors designed and constructed several chimeric antigen receptor (CAR) constructs, e.g., linking a single-chain fragment variant antigen-binding fragment of anti-OX40L-antibody with intracellular CD3zeta and 4-1BB or CD28 signaling domain. Also designed were constructs further linked to fluorescent protein reporter NeonGreen in order to visualize expression. In some of the constructs describe herein, construct expression is controlled by linking a Treg-specific promoter or by linking a non-specific MND promoter. These OX40L-CAR constructs have successfully transduced into Jurkat lymphoid cells and in human FACS-purified CD25+CD127− Treg cells (FIGS. 1A-1B and FIG. 7)). OX40L-CAR constructs express in a Treg specific manner and CAR expression does not interfere with ex vivo expansion of Tregs (FIGS. 1C-1D, FIGS. 2A-2D, FIGS. 8A-8B, and FIGS. 9A-9B). Stimulation of Treg cells transduced with OX40L-CAR constructs induces Treg activation markers at levels comparable to TCR stimulation (FIG. 3A). Furthermore, stimulation induces production of suppressive proteins such as IL-2, CTLA4, LAG-3, GARP, and LAP without inducing pro-inflammatory cytokine production such as IL17A, TNFa, and IFNg (FIG. 3B and FIGS. 10A-10B). The OX40L-CAR constructs can successfully mediate T-suppressive activity (FIG. 3C). These data demonstrate CAR-Treg cells remain functional and have T-suppressive functions. This functionality is illustrated in the model provided in FIG. 4.

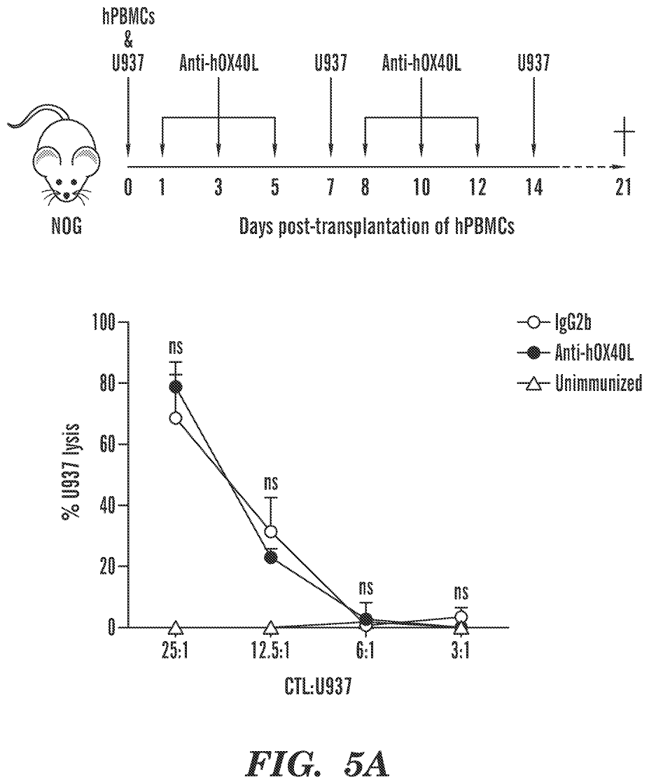

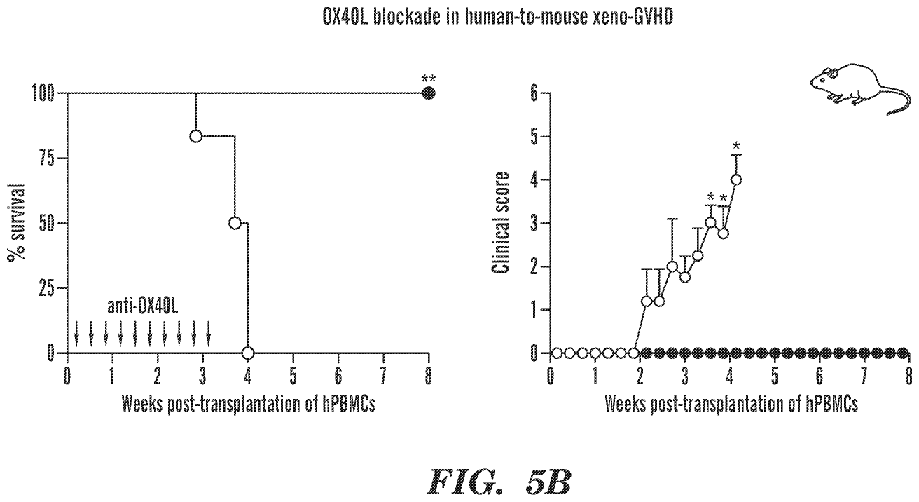

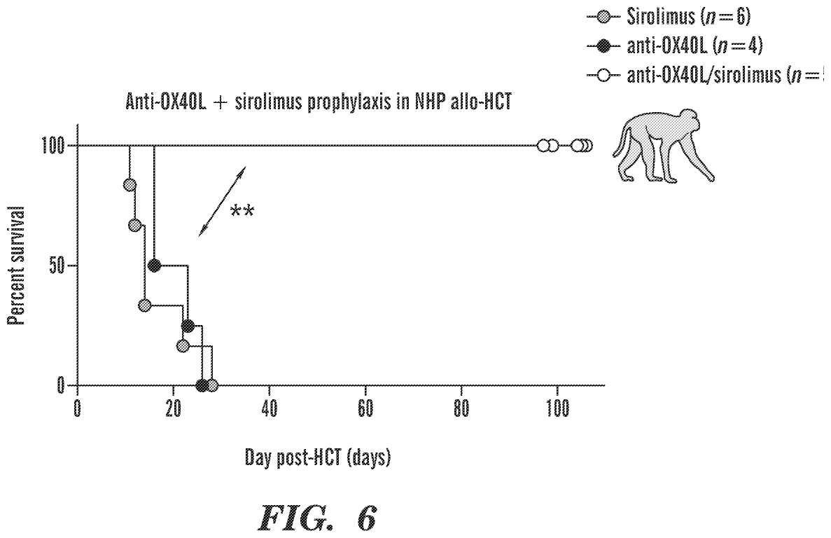

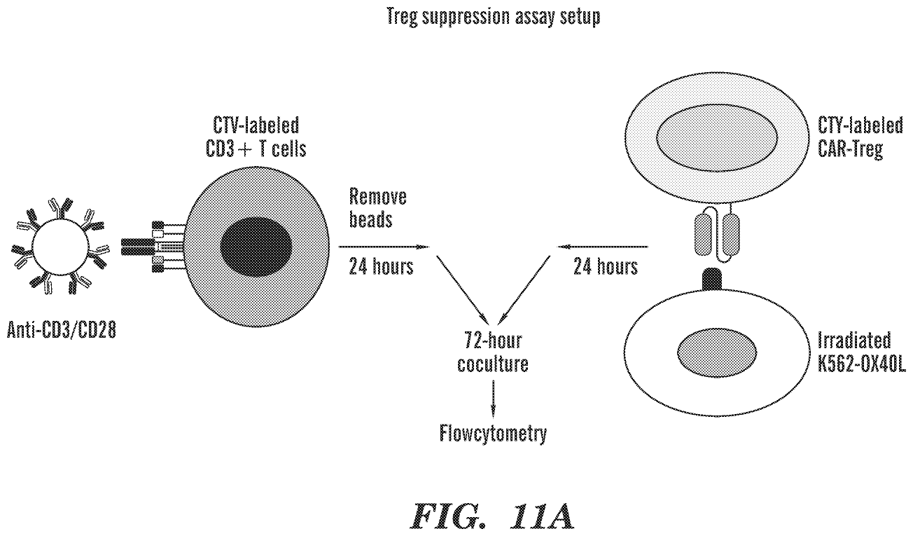

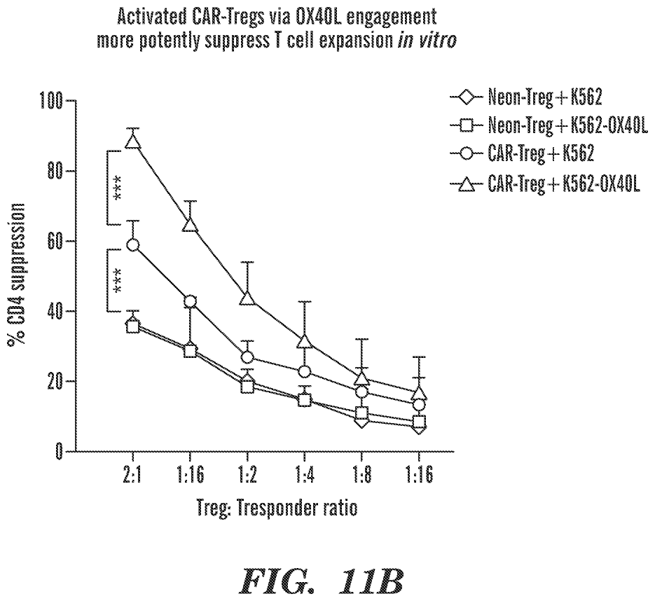

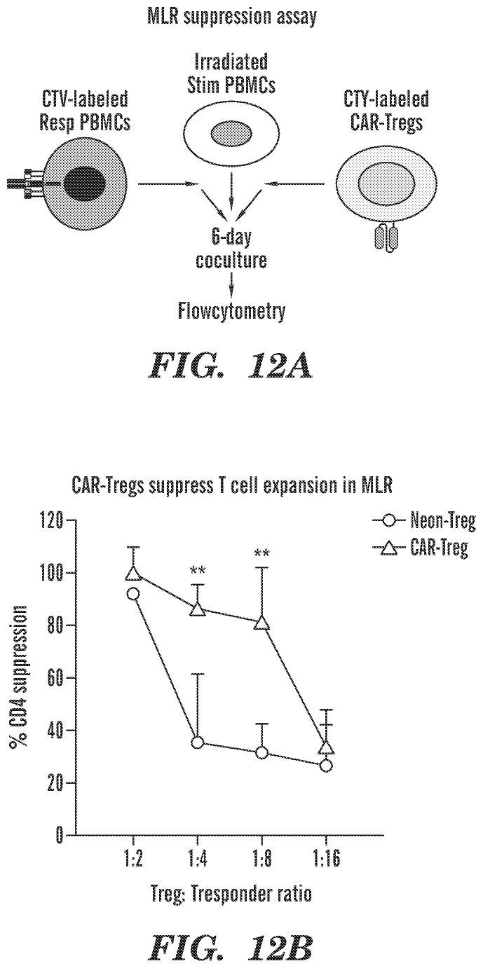

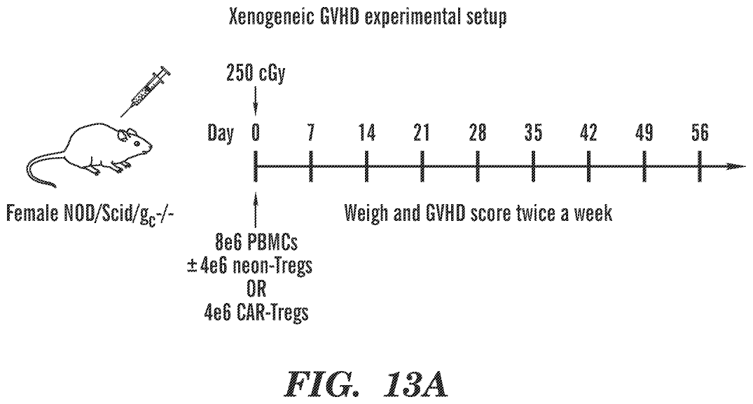

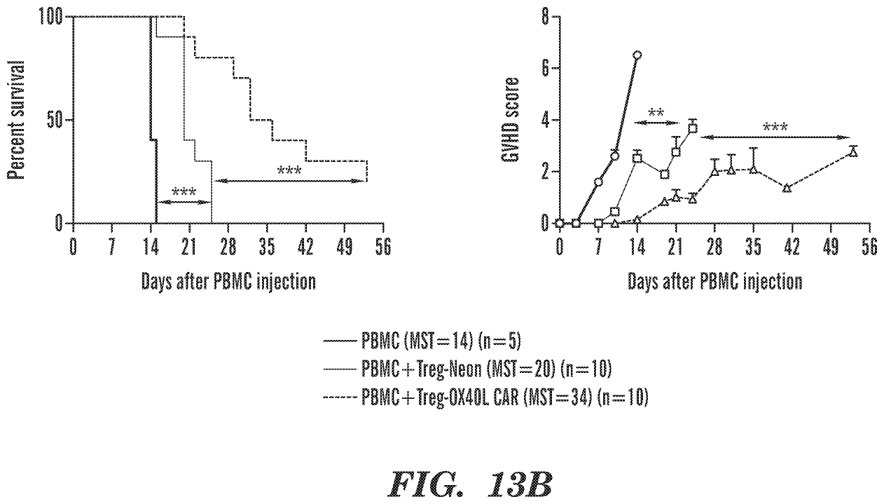

Blocking the interaction between OX40L and OX40 with anti-hOX40L antibody can effectively prevent and treat aGVHD by interfering with cytolytic activity of CD8+ T cells (FIGS. 5A-5B and FIG. 6). Demonstrated herein, OX40L-CAR-Tregs suppress T cell proliferation in vitro compared to Neon-Tregs. Furthermore, OX40L-CAR-Tregs demonstrated a greater capacity to inhibit monocyte-derived dendritic cell activation, highlighting the superior suppressive activity of these OX40L-CAR-Tregs compared to non-targeting Neon-Tregs (FIGS. 11A-11B and FIGS. 12A-12B). Importantly, in a human xenograft model of GvHD, induced by transplantation of human PBMC into sublethally irradiated NOD.Cg-Prkdc^{scid} Il2rg^{tmlWjl}/SzJ (NSG) mice, OX40L CAR-Treg demonstrated superior GvHD disease control compared to both PBMC alone and Neon-Tregs (FIGS. 13A-13B). Clinical scores were assessed by a combinatorial system including five parameters: weight loss, posture, mobility, skin, and fur conditions, and curves were compared using a 2-way ANOVA mixed-effects analysis, which demonstrated improvement in clinical xeno-GvHD with the OX40L CAR-Treg>Neon-Treg>PBMC alone over the entire length of analysis (p<0.001). An example of the substantial improvement in the clinical score on Day +14 is as follows: combined GvHD clinical score=6.5 for PBMC vs 2.5 for Neon-Treg vs 0.15 for OX40L CAR-Treg, p<0.0001 for all comparisons. This control of clinical GvHD resulted in prolonged recipient survival when compared to both PBMC alone and to control Neon-Tregs (MST=34 days for OX40L-CAR-Treg, 20 days for Neon-Treg, 14 days for PBMC alone, p<0.001).

Collectively, these results demonstrate a novel and efficacious approach using OX40L-CAR Tregs to enhance Treg suppressive function and to control GvHD. Because OX40L is upregulated on APCs in inflammatory environments, and its expression is limited to APCs and activated endothelial cells, this approach provides a unique strategy for the control of allo-immunity after HCT. Moreover, these OX40L-CAR Treg can be broadly applicable beyond HCT, to control allo-immunity after solid organ transplant and to suppress T cell activation in autoimmune diseases.

### Example 2

### FOXP3 Regulatory Elements Used for CAR-Tregs:

The endogenous FOXP3 promoter contains at least four highly conserved regulatory elements located across a large region of the FOXP3 gene [1]. These elements are identified as the FOXP3 core promoter sequence as well as three other conserved non-coding sequences (CNS 1-3). CNS1 is also known as the TGF-β sensor/enhancer, CNS2 is also known as Treg-cell-specific demethylation region (TSDR), and CNS3 is known as a FOXP3 pioneer element. These four regulatory elements play unique roles in the stabilization and tight control of FOXP3 expression in regulatory T (Treg) cells. When Treg cells receive signals from the TCR and CD28 stimulation, these signals induce activation of several transcription factors, including NFAT, c-Rel and AP-1, which bind to both the FOXP3 core promoter sequence and to CNS3 of the FOXP3 gene and initiate FOXP3 transcription. In contrast, CNS2 plays an essential role in the maintenance of FOXP3 expression. It contains CpG islands that are highly demethylated by the AML1/RUNX1 complex which promotes DNA demethylation of the region, thus this region is also known as TSDR. The demethylated TSDR further recruits STAT5 from IL-2 signaling and other transcription factors induced by TCR/CD28 stimulation to stabilize FOXP3 transcription. TGF-β induced SMAD2/3 gene expression, which is also actively involved in the FOXP3 gene expression, occurs by TGF-β binding to CNS1.

Because the natural FOXP3 promoter is too large to be directly utilized in a lentivirus vector as part of a gene expression cassette, the inventors employed a human synthetic FOXP3 promoter to selectively drive the OX40L CAR gene expression in Treg cells. The inventors designed this promoter using the same design previously published for restoring lineage-specific FoxP3 expression in mouse HSCs [2]. Finally, the inventors cloned all three CNS enhancer elements and placed them in tandem upstream of the FOXP3 core promoter sequence. In addition, the inventors included the FOXP3 5′- and 3′-untranslated regions (or UTRs) before and after the CAR gene cassette to maintain any potential post-transcriptional regulation of FOXP3 transcript. With the control of the synthetic human FOXP3 promoter, OX40L CAR protein is more selectively expressed on Treg cells rather than rare conventional T (Tcon) cells that could be expanded in small numbers during the Treg purification process.

### REFERENCES

1. Colamatteo A, Carbone F, Bruzzaniti S, Galgani M, Fusco C, Maniscalco G T, Di Rella F, de Candia P, De Rosa V. Molecular Mechanisms Controlling Foxp3 Expression in Health and Autoimmunity: From Epigenetic to Post-translational Regulation. Front Immunol. 2020; 10:3136.
2. Masiuk K E, Laborada J, Roncarolo M G, Hollis R P, Kohn D B. Lentiviral Gene Therapy in HSCs Restores Lineage-Specific Foxp3 Expression and Suppresses Autoimmunity in a Mouse Model of IPEX Syndrome. Cell Stem Cell. 2019; 24(2):309-317.e7

1. A chimeric antigen receptor (CAR) polypeptide comprising, from N-terminus to C-terminus:
a) an extracellular recognition portion that specifically binds to OX40L;
b) a transmembrane portion; and
c) an intracellular signaling portion.

2. The CAR of claim 1, further comprising, C-terminal of the intracellular signaling portion, a detectable polypeptide.

3. (canceled)

4. (canceled)

5. The CAR of claim 1, further comprising a cleavage site between the intracellular signaling portion and the detectable polypeptide.

6. (canceled)

7. The CAR of claim 1, wherein the recognition portion is an antibody reagent or ligand functional domain.

8. The CAR of claim 7, wherein the antibody reagent is a scFV.

9. The CAR of claim 7, wherein the antibody reagent is an anti-OX40L antibody reagent.

10. The CAR of claim 9, wherein the antibody reagent comprises CDR sequences at least 80% identical, at least 85% identical, at least 90% identical, at least 95% identical, or 100% identical to the six CDRs of SEQ ID NOs: 1-6.

11. The CAR of claim 9, wherein the antibody reagent comprises a sequence at least 80% identical, at least 85% identical, at least 90% identical, at least 95% identical, or 100% identical to the amino acid sequences of SEQ ID NO 7-12.

12. The CAR of claim 1, wherein the intracellular signaling portion comprises one or more of a CD28 co-signaling domain, a 41BB co-signaling domain, a IL2Rα JAK3 and IL2Rβ STAT5 composite docking site, a TGFβ-R SMAD2/3 docking site, and a CD3zeta signaling domain.

13. (canceled)

14. (canceled)

15. A nucleic acid molecule encoding the CAR of claim 1.

16. The nucleic acid molecule of claim 15, wherein the expression of the CAR is controlled by a Treg-specific promoter or a MND promoter.

17. (canceled)

18. A vector comprising the nucleic acid molecule of claim 15.

19. A cell comprising the CAR of claim 1.

20. The cell of claim 19, wherein the cell is a Treg.

21. (canceled)

22. (canceled)

23. A population of cells, at least 80% of which are cells according to claim 19.

24. A method of treating an autoimmune or inflammatory condition in a subject in need thereof, the method comprising administering to the subject a cell of claim 19.

25. The method of claim 24, wherein said autoimmune or inflammatory condition comprises an allograft rejection, xenograft rejection, or graft-vs. host disease (GVHD).

26. The method of claim 24, wherein said autoimmune or inflammatory condition is selected from the group consisting of an inflammatory bowel disease; rheumatoid arthritis; type I diabetes mellitus or autoimmune insulitis; multiple sclerosis; autoimmune thyroiditis; autoimmune gastritis; autoimmune uveitis or uveoretinitis; autoimmune orchitis; autoimmune oophoritis; psoriasis; vitiligo; autoimmune prostatitis; any undesired immune response; tissue rejection; and an inflammatory condition.

27. The method of claim 24, wherein the population of Tregs are autologous to the subject.

28. The method of claim 24, wherein the population of Tregs are allogenic to the subject.

29. (canceled)

30. (canceled)

31. (canceled)

32. (canceled)

33. (canceled)
